(19) United States
(12) Patent Application Publication
(10) Pub. No.: US 2016/0319277 A1
(43) Pub. Date: Nov. 3, 2016

(54) METHODS OF IMPROVED PROTEIN PRODUCTION USING MIRNAs AND SIRNAs

(71) Applicants: THE JOHNS HOPKINS UNIVERSITY, Baltimore, MD (US); NATIONAL INSTITUTES OF HEALTH, Bethesda, MD (US); NOVARTIS INSTITUTES OF BIOMEDICAL RESEARCH, INC., Cambridge, MA (US)

(72) Inventors: Joseph Shiloach, Bethesda, MD (US); Michael J. Betenbaugh, Baltimore, MD (US); Scott E. Martin, Rockville, MD (US); Su Xiao, Bethesda, MD (US); Yu-Chi Chen, Cambridge, MA (US)

(21) Appl. No.: 15/009,481

(22) Filed: Jan. 28, 2016

Related U.S. Application Data
(60) Provisional application No. 62/108,976, filed on Jan. 28, 2015.

(51) Int. Cl. C12N 15/113; C12Q 1/68

(52) U.S. Cl. CPC C12N 15/113; C12Q 1/6876; C12N 2310/14; C12N 2310/141; C12Q 2600/178; C12Q 2600/158

(57) ABSTRACT

The invention provides a method of increasing protein production in a cell by contacting the cell with miRNA, siRNA or a combination thereof, or increasing protein production by genome editing methodologies to silence or inhibit gene expression. A screening method for obtaining such miRNA or siRNA species is also provided, as well as identification of target genes for genome editing.

### RELATED APPLICATION DATA

This application claims the benefit of priority under 35 U.S.C. §119(e) of U.S. Provisional Patent Application Ser. No. 62/108,976, filed Jan. 28, 2015, the entire contents of which is incorporated herein by reference in its entirety.

### STATEMENT OF GOVERNMENT SUPPORT

This invention was also made in part with government support under Grant No. DK075080-04 awarded by the Intramural Research Program of the National Institute of Diabetes and Digestive and Kidney Diseases (NIDDK/NIH), National Center for Advancing Translational Sciences (NCATS/NIH) and the National Institute of Dental and Craniofacial Research (NIDCR/NIH). The United States government has certain rights in this invention.

### BACKGROUND OF THE INVENTION

1. Field of the Invention

The present invention relates generally to recombinant protein production, and more specifically to methods for increased protein production using miRNAs and siRNAs as well as compositions utilized in such methods.

2. Background Information

Improving the expression level of recombinant mammalian proteins has not only been pursued by biotechnologist for production of commercial biotherapeutics, but has also been at the heart of numerous biomedical studies in academia, as an adequate supply of correctly folded proteins is a prerequisite for all structure and function studies. A critical area is mammalian integral membrane proteins such as receptors, ion channels and transporters which are encoded by 20-40% of all Open Reading Frames (ORFs) in the mammalian genome and are targets of most of the medicines sold worldwide. Even though more than 100,000 structures have been deposited in Protein Data bank, the overexpression of membrane protein remains difficult and only 898 membrane protein structures are available as of Oct. 2, 2014. Rational attempts to improve membrane protein expression may not lead to expected results as membrane proteins involve particularly complex folding, assembly, and processing pathways, and there is only limited information for the bottlenecks that may reside in the protein production steps, such as transcription, translation, protein folding, secretion and cell viability.

MiRNAs have emerged as powerful tools for engineering cells with desirable properties, such as improved protein production capabilities and enhanced anti-apoptotic properties under stress conditions. MiRNAs are a novel class of small, non-coding RNAs that can simultaneously silence multiple genes by binding to their 3′-untranslated regions (3′-UTR). They exhibit a broad spectrum of regulatory effects in eukaryotic cellular processes including cell growth and apoptosis, cell differentiation and metabolism, cancer development and progression. Their capacity to globally regulate entire gene networks and not introduce an additional translational burden (compared to gene overexpression strategies) makes them particularly advantageous for cell line development.

MicroRNAs (miRNAs) are approximately 21 nucleotide single-stranded small RNAs that regulate posttranscriptional gene expression in metazoans and plants. miRNAs are processed from hairpin precursors and assembled into functional complexes containing Argonaute proteins (termed RNA-induced silencing complex (RISC)), which suppress target mRNA expression. miRNAs are usually generated from noncoding regions of gene transcripts and function to suppress gene expression by translational repression and/or by enhancing mRNA destabilization RNA degradation. Mature microRNAs are short, single-stranded RNA molecules approximately 22 nucleotides in length. MicroRNAs are sometimes encoded by multiple loci, some of which are organized in tandemly co-transcribed clusters.

MicroRNAs usually induce gene silencing by binding to target sites found within the 3′UTR of the targeted mRNA. This interaction prevents protein production by suppressing protein synthesis and/or by initiating mRNA degradation. Since most target sites on the mRNA have only partial base complementarity with their corresponding microRNA, individual microRNAs may target as many as 100 different mRNAs. Moreover, individual mRNAs may contain multiple binding sites for different microRNAs, resulting in a complex regulatory network.

MicroRNAs have been shown to be involved in a wide range of biological processes such as cell cycle control, apoptosis and several developmental and physiological processes including stem cell differentiation, hematopoiesis, hypoxia, cardiac and skeletal muscle development, neurogenesis, insulin secretion, cholesterol metabolism, aging, immune responses and viral replication. In addition, highly tissue-specific expression and distinct temporal expression patterns during embryogenesis suggest that microRNAs play a key role in the differentiation and maintenance of tissue identity.

In recent years, miRNAs have emerged as regulators of numerous activities, including developmental processes, disease pathogenesis, and host-pathogen interactions. miRNA expression and gene regulation is a wide-spread phenomenon, and according to recent miRNA annotation and deep-sequencing data, there are >15,000 microRNA gene loci spanning >140 species and >17,000 distinct mature microRNA sequences. These numbers will surely increase as high-throughput RNA sequencing technologies are applied to discovery of new non-coding RNA.

RNA interference (RNAi), first discovered as a natural biological process of eukaryotic cells for protecting the genome against foreign nucleic acids, has been developed and utilized as a revolutionary tool in deducing gene functions and in combating genetic defects, viral diseases, autoimmune disorders, and cancers. siRNAs are 21-25 nucleotide double-strand RNA fragments with symmetric 2-nucleotides 3′-end overhangs. The guide strand of siRNA can be incorporated into RNA-induced silencing complex (RISC), which brings about sequence-specific degradation of the homologous single stranded mRNAs. In recent years, large-scale genetic screens have been made possible by the availability of genome-wide siRNA libraries, as well as the development of sophisticated new instrumentation and bioinformatics approaches for data analysis. They have been used to investigate the biological functions of specific genes and pathways in various diseases and important biological processes, including signal transduction, cell aging or death, cell or organelle organization, protein localization and responses of host cells to pathogens. However, there has been limited use of a genome-wide siRNA screen for improving heterologous protein production, an important process intensively investigated by the pharmaceutical and biotechnology industry.

### SUMMARY OF THE INVENTION

The present invention is based on the discovery of miRNAs and siRNAs that can be utilized, either alone or in combination, to enhance protein production.

In one embodiment, the invention provides a method of increasing production of a protein of interest in a cell. The method includes contacting the cell with an miRNA, siRNA or combination thereof under conditions wherein the miRNA or siRNA is incorporated into the cell, wherein an increase in production of the protein greater than that of a control cell not contacted with the miRNA or siRNA is indicative of increased protein production in the cell, thereby increasing production of the protein of interest in the cell. In one aspect, the cell is a mammalian cell, for example, an HEK or CHO cell. In one aspect, the cell transiently expresses the protein and in one aspect, the cell stably expresses the protein. The protein, can be a cytosolic, secreted or a membrane protein, for example.

In another embodiment, the invention provides miRNAs and siRNAs for use in increasing protein production.

In yet another embodiment, the invention provides a vector including the miRNA or siRNA of the invention.

In still another embodiment, the invention provides a cell which includes in the vector of the invention.

In a further embodiment, the invention provides a kit for increasing protein production in a cell. The kit includes a miRNA of the present invention, e.g., a miRNA sequence having a sequence as set forth in SEQ ID NOs:1-26, and a siRNA which inhibits expression of a gene set forth in Table 3.

In yet another embodiment, the invention provides a kit including a reagent for inhibiting or silencing a gene listed in Table 3 for increasing protein production in a cell. In embodiments, the reagent is used to accomplish a genome editing methodology including, but not limited to, a Crispr, zinc finger nuclease, or transcription activator-like effector nuclease (Talen).

In still another embodiment, the invention further provides a method of increasing production of a protein of interest in a cell comprising inhibiting or silencing one or more genes as listed in Table 3. In embodiments, silencing or inhibition is achieved via a genome editing methodology, for example a methodology that includes use of a Crispr, zinc finger nuclease, or transcription activator-like effector nuclease (Talen). In some embodiments, expression of the gene is knocked-out or knocked-down. In some embodiments, silencing or inhibition of gene expression results from deletion or mutation of the gene.

In another embodiment, the invention provides a screening method for obtaining miRNAs for enhancing expression of a protein. The method includes: a) contacting a cell comprising a detectably labeled protein with a plurality of miRNAs; and b) measuring protein production prior to and after contacting with the miRNAs, wherein an increase in expression of the protein after contact is indicative of an miRNA for enhancing expression of the protein. In one aspect, the invention provides for assessing the functionality of the enhanced protein produced.

### BRIEF DESCRIPTION OF THE DRAWINGS

FIGS. 1A-1C are graphical representations of data relating to a miRNA screen with a stable T-REx-293-NTSR1-GFP cell line. FIG. 1A is a graphical representation of data relating to a miRNA screen with a stable T-REx-293-NTSR1-GFP cell line. FIG. 1B is a graphical representation of data relating to a miRNA screen with a stable T-REx-293-NTSR1-GFP cell line. Figure C is a graphical representation of data relating to a miRNA screen with a stable T-REx-293-NTSR1-GFP cell line.

FIGS. 2A-2C are graphical representations of data relating to flow cytometry analysis on T-REx-293-NTSR1-GFP cells transfected with 26 miRNAs. FIG. 2A is a graphical representation of data relating to flow cytometry analysis on T-REx-293-NTSR1-GFP cells transfected with 26 miRNAs. FIG. 2B is a graphical representation of data relating to flow cytometry analysis on T-Rex-293-NTSRI-GFP cell line. FIG. 2C is a graphical representation of data relating to flow cytometry analysis on T-Rex-293-NTSRI-GFP cell line.

FIGS. 3A-3B are graphical representations of data relating to validation of improved functional expression of NTSR1 with a [³H]NT binding assay. FIG. 3A is a graphical representation of data relating to validation of improved functional expression of NTSR1 with a [3H]NT binding assay. FIG. 3B is a graphical representation of data relating to validation of improved functional expression of NTSR1 with a [3H]NT binding assay.

FIGS. 4A-4C are graphical and tabular representations of data relating to a miRNA screen with a stable HEK-CMV-Luc2-Hygro cell line. FIG. 4A is a schematic representation of relating to a miRNA screen with a stable HEK-CMV-Luc2-Hygro cell line. FIG. 4B is a graphical representation of data relating to a miRNA screen with a stable HEK-CMV-Luc2-Hygro cell line. FIG. 4C is a tabular representation of data relating to a miRNA screen with a stable HEK-CMV-Luc2-Hygro cell line and shows the following miRNA sequences: miR-22-5p (SEQ ID NO:3); miR-221-5p (SEQ ID NO:1); miR-892b (SEQ ID NO:4); miR-18a-5p (SEQ ID NO:25); miR-22-3p (SEQ ID NO:21); miR-429 (SEQ ID NO:2); and miR-2110 (SEQ ID NO:20).

FIGS. 5A-5C are graphical representations of data relating to validation of improved luciferase activity. FIG. 5A is a graphical representation of data relating to validation of improved luciferase activity. FIG. 5B is a graphical representation of data relating to validation of improved luciferase activity. Figure C is a graphical representation of data relating to validation of improved luciferase activity.

FIGS. 6A-6C are graphical representations of data showing improved glypican-3(GPC3) hFc-fusion protein secretion by five miRNAs. FIG. 6A is a graphical representation of data showing improved glypican-3(GPC3) hFc-fusion protein secretion by five miRNAs. FIG. 6C is a graphical representation of data showing improved glypican-3(GPC3) hFc-fusion protein secretion by five miRNAs. FIG. 6B is a graphical representation of data showing improved glypican-3(GPC3) hFc-fusion protein secretion by five miRNAs. FIG. 6C is a graphical representation of data showing improved glypican-3(GPC3) hFc-fusion protein secretion by five miRNAs.

FIG. 7 is a pictorial diagram of a plasmid map for pJMA-NTSR1-GFP.

FIGS. 8A-8C are pictorial and graphical representations of data relating to a genome-wide human siRNA library screen with HEK-CMV-luc2-Hygro cell line. FIG. 8A is a pictorial representation of data relating to a genome-wide human siRNA library screen with HEK-CMV-luc2-Hygro cell line. FIG. 8B is a graphical representation of data relating to a genome-wide human siRNA library screen with HEK-CMV-luc2-Hygro cell line. FIG. 8C is a graphical representation of data relating to a genome-wide human siRNA library screen with HEK-CMV-luc2-Hygro cell line.

FIG. 9 is a graphical representation relating to the functional categorization of strong enhancer siRNA-associated genes.

FIGS. 10A-10D are graphical representations of data regarding the effects of 10 selected enhancer siRNAs on four HEK cell lines expressing different recombinant proteins. FIG. 10A is a graphical representation of data regarding the effects of 10 selected enhancer siRNAs on four HEK cell lines expressing different recombinant proteins. FIG. 10B is a graphical representation of data regarding the effects of 10 selected enhancer siRNAs on four HEK cell lines expressing different recombinant proteins. FIG. 10C is a graphical representation of data regarding the effects of 10 selected enhancer siRNAs on four HEK cell lines expressing different recombinant proteins. FIG. 10D is a graphical representation of data regarding the effects of 10 selected enhancer siRNAs on four HEK cell lines expressing different recombinant proteins.

FIGS. 11A-11C are graphical representations of data depicting a time course of the effects of OAZ1siRNA transfection on cell viability and luciferase yield, and the mRNA levels of OAZ1 and luciferase. FIG. 11A is a graphical representation of data depicting a time course of the effects of OAZ1siRNA transfection on cell viability and luciferase yield, and the mRNA levels of OAZ1 and luciferase. FIG. 11B is a graphical representation of data depicting a time course of the effects of OAZ1siRNA transfection on cell viability and luciferase yield, and the mRNA levels of OAZ1 and luciferase. FIG. 11C is a graphical representation of data depicting a time course of the effects of OAZ1siRNA transfection on cell viability and luciferase yield, and the mRNA levels of OAZ1 and luciferase.

FIGS. 12A-12C are graphical representations of data depicting a time course of the effects of OAZ1 silencing on the levels of ODC protein, ODC mRNA and cellular polyamines. FIG. 12A is a graphical representation of data depicting a time course of the effects of OAZ1 silencing on the levels of ODC protein, ODC mRNA and cellular polyamines. FIG. 12B is a graphical representation of data depicting a time course of the effects of OAZ1 silencing on the levels of ODC protein, ODC mRNA and cellular polyamines. FIG. 12C is a graphical representations of data depicting a time course of the effects of OAZ1 silencing on the levels of ODC protein, ODC mRNA and cellular polyamines.

FIGS. 13A-13C are graphical representations of data depicting the effect of exogenous polyamines on luciferase expression and cell growth. FIG. 13A is a graphical representation of data depicting the effect of exogenous polyamines on luciferase expression and cell growth. FIG. 13B is a graphical representation of data depicting the effect of exogenous polyamines on luciferase expression and cell growth. FIG. 13C is a graphical representation of data depicting the effect of exogenous polyamines on luciferase expression and cell growth.

FIG. 14 is a schematic diagram of the polyamine pathway and regulation of ornithine decarboxylase (ODC) by antizyme (OAZ) and antizyme inhibitor (AZIN).

### DETAILED DESCRIPTION OF THE INVENTION

The present invention is based on the seminal discovery of miRNAs and siRNAs that enhance protein production in a cell. The miRNAs and siRNAs may be used alone or in combination to increase cellular protein production of a protein of interest.

Before the present methods are described, it is to be understood that this invention is not limited to particular methods, and experimental conditions described, as such methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Obtaining adequate quantities of functional mammalian membrane proteins has been a bottleneck in their structural and functional studies because the expression of these proteins from mammalian cells is relatively low. To explore the possibility of enhancing expression of these proteins using miRNA, a stable T-REx-293 cell line expressing the neurotensin receptor type 1 (NTSR1), a hard-to-express G protein-coupled receptor (GPCR), was constructed. The cell line was then subjected to human miRNA mimic library screening. In parallel, an HEK293 cell line expressing luciferase was also screened with the same human miRNA mimic library. Five microRNA mimics: hsa-miR-22-5p (SEQ ID NO:3), hsa-miR-18a-5p (SEQ ID NO:25), hsa-miR-22-3p (SEQ ID NO:21), hsa-miR-429 (SEQ ID NO:2) and hsa-miR-2110 (SEQ ID NO:20) were identified from both screens. They led to 48% increase in the expression of functional NTSR1 and to 239% increase of luciferase expression. These miRNAs were also effective in enhancing the expression of secreted glypican-3 hFc-fusion protein in HEK293 cell. The results indicate that these molecules may have a wide role in enhancing production of proteins with biomedical interest.

In a related aspect, for the purpose of improving recombinant protein production from mammalian cells, an unbiased, high-throughput whole-genome RNA interference screen was conducted using human embryonic kidney 293 (HEK 293) cells expressing firefly luciferase. 21,585 human genes were individually silenced with three different siRNAs for each gene. 56 genes whose silencing caused the greatest improvement in the luciferase expression were found to be part of several different pathways that are associated with spliceosome formation/mRNA processing, transcription, metabolic process, transport and protein folding. 10 genes whose downregulation significantly enhanced the protein expression were validated by their silencing effect on four different recombinant proteins. Among the validated genes, OAZ1—the gene encoding the ornithine decarboxylase antizyme1—was selected for detailed investigation, since its silencing improved the reporter protein production without affecting cell viability. Silencing OAZ1 caused the increase of the omithine decarboxylase enzyme and the cellular levels of putrescine and spermidine, and indicated that increased cellular polyamines enhanced luciferase expression without affecting its transcription. The study shows that OAZ1 is a novel target for improving expression of recombinant proteins. The genome-scale screening demonstrated in this work can establish the foundation for targeted design of an efficient mammalian cell platform for different biotechnological applications.

### DEFINITIONS

The terms “microRNA”, “miRNA”, or “miR” all refer to non-coding RNAs (and also, as the context will indicate, to DNA sequences that encode such RNAs) that are capable of entering the RNAi pathway and regulating gene expression. “Primary miRNA” or “pri-miRNA” represents the non-coding transcript prior to Drosha processing and includes the stem-loop structure(s) as well as flanking 5′ and 3′ sequences. “Precursor miRNAs” or “pre-miRNA” represents the non-coding transcript after Drosha processing of the pri-miRNA. The term “mature miRNA” can refer to the double stranded product resulting from Dicer processing of pre-miRNA or the single stranded product that is introduced into RISC following Dicer processing. In some cases, only a single strand of an miRNA enters the RNAi pathway. In other cases, two strands of a miRNA are capable of entering the RNAi pathway. Illustrative examples of the invention are provided in Attachment A.

As used herein, the term “RNA silencing” refers to a group of sequence-specific regulatory mechanisms (e.g., RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target” polynucleotide sequence”, e.g. when both polynucleotide sequences are present in the same cell. In certain embodiments, the target polynucleotide sequence corresponds to a target gene, while the non-target polynucleotide sequence corresponds to a non-target gene. In other embodiments, the target polynucleotide sequence corresponds to a target allele, while the non-target polynucleotide sequence corresponds to a non-target allele. In certain embodiments, the target polynucleotide sequence is the DNA sequence encoding the regulatory region (e.g. promoter or enhancer elements) of a target gene. In other embodiments, the target polynucleotide sequence is a target mRNA encoded by a target gene.

As used herein, the term “target gene” is a gene whose expression is to be substantially inhibited or “silenced” as regards siRNA, or a gene or plurality of genes that serve as regulatory genes in which increased expression results in increased protein production as regards miRNA. This silencing can be achieved by RNA silencing, for example by cleaving the mRNA of the target gene or by translational repression of the target gene. Alternatively, inhibition or silencing may be achieved by genome editing tools to inhibit expression of the gene at the genomic level, e.g., gene knock-out via, for example, deletion or mutation of the gene. The term “non-target gene” is a gene whose expression is not to be substantially inhibited. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms. In another embodiment, the target and non-target genes can share less than 100% sequence identity. In another embodiment, the non-target gene may be a homolog (e.g. an ortholog or paralog) of the target gene.

A “target allele” is an allele whose expression is to be selectively inhibited or “silenced.” This silencing can be achieved by RNA silencing, such as, for example, by cleaving the mRNA of the target gene or target allele by an siRNA. Alternatively, inhibition may be achieved by genome editing tools to inhibit expression of the gene at the genomic level, e.g., gene knock-out via, for example, deletion or mutation of the gene. The term “non-target allele” is a allele whose expression is not to be substantially inhibited. In certain embodiments, the target and non-target alleles can correspond to the same target gene. In other embodiments, the target allele corresponds to a target gene, and the non-target allele corresponds to a non-target gene. In one embodiment, the polynucleotide sequences of the target and non-target alleles can differ by one or more nucleotides. In another embodiment, the target and non-target alleles can differ by one or more allelic polymorphisms. In another embodiment, the target and non-target alleles can share less than 100% sequence identity.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, and dual-function oligonucleotides as well as precursors thereof. In a certain embodiment, the RNA silencing agent is capable of silencing miRNA either by an RNA-induced silencing complex (RISC)-like ribonucleoprotein particle (miRNP) which inhibits translations or, depending on the degree of Watson-Crick complementarity, induces degradation of target mRNAs. In another embodiment, the RNA silencing agent is capable of inducing RNA interference (RNAi). In yet another embodiment, the RNA silencing agent is capable of mediating translational repression.

As used herein, the term “microRNA inhibitor” or “anti-microRNA” is synonymous with the term “microRNA antagonist”. Additionally, the term “microRNA mimic” is synonymous with the term “microRNA agonist”.

The term “nucleoside” refers to a molecule having a purine or pyrimidine base. covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, ²N-methylguanosine and ^{2,2}N,N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g. by DNA replication or transcription of DNA, respectively). RNA can be post-transcriptionally modified. DNA and RNA can also be chemically synthesized. DNA and RNA can be single-stranded (i.e. ssRNA and ssDNA, respectively) or multi-stranded (e.g. double stranded, i.e. dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “rare nucleotide” refers to a naturally occurring nucleotide that occurs infrequently, including naturally occurring deoxyribonucleotides or ribonucleotides that occur infrequently, e.g. a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and ^{2,2}N,N-dimethylguanosine.

The term “nucleotide analog” or “altered nucleotide” or “modified nucleotide” refers to a non-standard nucleotide, including non-naturally occurring ribonucleotides or deoxyribonucleotides. Nucleotide analogs may be modified at any position so as to alter certain chemical properties of the nucleotide yet retain the ability of the nucleotideanalog to perform its intended function. Examples of modified nucleotides include, but are not limited to, 2-amino-guanosine, 2-amino-adenosine, 2,6-diamino-guanosine and 2,6-diamino-adenosine. Examples of positions of the nucleotide which may be derivitized include the 5 position, e.g. 5-(2-amino)propyl uridine, 5-bromo uridine, 5-propyne uridine, 5-propenyl uridine, etc.; the 6 position, e.g. 6-(2-amino)propyl uridine; the 8-position for adenosine and/or guanosines, e.g. 8-bromo guanosine, 8-chloro guanosine, 8-fluoroguanosine, and the like.

Nucleotide analogs also include deaza nucleotides, e.g. 7-deaza-adenosine; O- and N-modified (e.g. alkylated, e.g. N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C1-C6 alkyl, alkenyl, alkynyl, aryl, and the like. Other possible modifications include those described in U.S. Pat. Nos. 5,858,988, and 6,291,438.

The phosphate group of the nucleotide may also be modified, e.g. by substituting one or more of the oxygens of the phosphate group with sulfur (e.g. phosphorothioates), or by making other substitutions which allow the nucleotide to perform its intended function such as described in, for example, Eckstein, Antisense Nucleic Acid Drug Dev. 2000 Apr. 10(2): 117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic AcidDrug Dev. 2001 Apr. 11(2):77-85, and U.S. Pat. No. 5,684,143. Certain of the above-referenced modifications (e.g. phosphate group modifications) decrease the rate of hydrolysis of, for example, polynucleotides comprising the analogs in vivo or in vitro.

The term “oligonucleotide” refers to a short polymer of nucleotides and/or nucleotide analogs. The term “RNA analog” refers to a polynucleotide (e.g. a chemically synthesized polynucleotide) having at least one altered or modified nucleotide as compared to a corresponding unaltered or unmodified RNA but retaining the same or similar nature or function as the corresponding unaltered or unmodified RNA. The oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, and/or phosphorothioate linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA silencing (e.g. RNA interference). In an exemplary embodiment, oligonucleotides comprise Locked Nucleic Acids (LNAs) or Peptide Nucleic Acids (PNAs).

As used here, the term “melting temperature” or “Tm” refers to the temperature at which half of a population of double-stranded polynucleotide molecules becomes dissociated into single strands.

As used herein, the terms “sufficient complementarity” or “sufficient degree of complementarity” mean that the RNA silencing agent has a sequence (e.g. in the antisense strand, mRNA targeting moiety or miRNA recruiting moiety) which is sufficient to bind the desired target RNA respectively, and to trigger the RNA silencing of the target mRNA.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

As used herein, the term “small interfering RNA” (“siRNA”) (also referred to in the art as “short interfering RNAs”) refers to an RNA (or RNA analog) comprising between about 5-60 nucleotides (or nucleotide analogs) which is capable of directing or mediating RNA silencing (e.g. RNA interference or translational repression). A siRNA may comprise between about 15-30 nucleotides or nucleotide analogs, between about 16-25 nucleotides (or nucleotide analogs), between about 18-23 nucleotides (or nucleotide analogs), and between about 19-22 nucleotides (or nucleotide analogs) (e.g. 19, 20, 21 or 22 nucleotides or nucleotide analogs). The term “short” siRNA refers to a siRNA comprising 5-23 nucleotides, ˜21 nucleotides (or nucleotide analogs), for example, 19, 20, 21 or 22 nucleotides. The term “long” siRNA refers to a siRNA comprising 24-60 nucleotides, ˜24-25 nucleotides, for example, 23, 24, 25 or 26 nucleotides. Short siRNAs may, in some instances, include fewer than 19 nucleotides, e.g. 16, 17 or 18 nucleotides, or as few as 5 nucleotides, provided that the shorter siRNA retains the ability to mediate RNAi. Likewise, long siRNAs may, in some instances, include more than 26 nucleotides, e.g. 27, 28, 29, 30, 35, 40, 45, 50, 55, or even 60 nucleotides, provided that the longer siRNA retains the ability to mediate RNAi or translational repression absent further processing, e.g. enzymatic processing, to a short siRNA.

As used herein, the term “antisense strand” of an RNA silencing agent, e.g. an siRNA or RNAi agent, refers to a strand that is substantially complementary to a section of about 10-50 nucleotides, e.g. about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g. complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g. an siRNA or RNAi agent, refers to a strand that is complementary to the antisense strand or first strand. Antisense and sense strands can also be referred to as first or second strands, the first or second strand having complementarity to the target sequence and the respective second or first strand having complementarity to the first or second strand. miRNA duplex intermediates or siRNA-like duplexes include a miRNA strand having sufficient complementarity to a section of about 10-50 nucleotides of the mRNA of the gene targeted for silencing and a miRNA strand having sufficient complementarity to form a duplex with the miRNA strand.

As used herein, the term “guide strand” refers to a strand of an RNAi agent, e.g. an antisense strand of an miRNA duplex or miRNA sequence, that enters into the RISC complex and directs cleavage of the target mRNA.

As used herein, the term “passenger strand” refers to the strand typically not incorporated into risk, present in lower levels in the steady state. It is to be understood, however, that in certain cases, both strands of the duplex, i.e., both the “passenger strand” and the “guide strand” are viable and may be functional miRNA that enters into the RISC complex and directs cleavage of the target mRNA;

The term “engineered,” as in an engineered RNA precursor, or an engineered nucleic acid molecule, indicates that the precursor or molecule is not found in nature, in that all or a portion of the nucleic acid sequence of the precursor or molecule is created or selected by man. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

An “isolated nucleic acid molecule or sequence” is a nucleic acid molecule or sequence that is not immediately contiguous with both of the coding sequences with which it is immediately contiguous (one on the 5′ end and one on the 3′ end) in the naturally occurring genome of the organism from which it is derived. The term therefore includes, for example, a recombinant DNA or RNA that is incorporated into a vector; into an autonomously replicating plasmid or virus; or into the genomic DNA of a prokaryote or eukaryote, or which exists as a separate molecule (e.g. a cDNA or a genomic DNA fragment produced by PCR or restriction endonuclease treatment) independent of other sequences. It also includes a recombinant DNA that is part of a hybrid gene encoding an additional polypeptide sequence.

As used herein, the term “isolated RNA” (e.g. “isolated shRNA”, “isolated siRNA”, “isolated siRNA-like duplex”, “isolated miRNA”, “isolated gene silencing agent”, or “isolated RNAi agent”) refers to RNA molecules which are substantially free of other cellular material, or culture medium when produced by recombinant techniques, or substantially free of chemical precursors or other chemicals when chemically synthesized.

As used herein, the term “transgene” refers to any nucleic acid molecule, which is inserted by artifice into a cell, and becomes part of the genome of the organism that develops from the cell. Such a transgene may include a gene that is partly or entirely heterologous (i.e. foreign) to the transgenic organism, or may represent a gene homologous to an endogenous gene of the organism. The term “transgene” also means a nucleic acid molecule that includes one or more selected nucleic acid sequences, e.g. DNAs, that encode one or more engineered RNA precursors, to be expressed in a transgenic organism, e.g. animal, which is partly or entirely heterologous, i.e. foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected nucleic acid sequence, all operably linked to the selected sequence, and may include an enhancer sequence.

As used herein, “silencing” or “inhibiting” refers to various methods to reduce or eliminate expression of a target gene using siRNA as well as genome editing including CRisprs, zinc fingers, and tale nucleases. Such methods are used to knock-out or knock-down a gene.

A gene “involved” in a disease or disorder includes a gene, the normal or aberrant expression or function of which effects or causes the disease or disorder or at least one symptom of the disease or disorder.

Sequence identity may be determined by sequence comparison and alignment algorithms known in the art. To determine the percent identity of two nucleic acid sequences (or of two amino acid sequences), the sequences are aligned for optimal comparison purposes (e.g. gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, the molecules are identical at that position. The percent identity between the two sequences is a function of the number of identical positions shared by the sequences (i.e. % homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e. a local alignment). A non-limiting example of a local alignment algorithm utilized for the comparison of sequences is the algorithm of Karlin and Altschul (1990) Proc. Natl. Acad Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e. a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e. a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

miRNAs are noncoding RNAs of approximately 22 nucleotides which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof.

The miRNA sequence can be similar or identical to that of any naturally occurring miRNA (see e.g. The miRNA Registry; Griffiths-Jones S, Nuc. Acids Res., 2004). Over one thousand natural miRNAs have been identified to date and together they are thought to comprise ˜1% of all predicted genes in the genome. Many natural miRNAs are clustered together in the introns of pre-mRNAs and can be identified in silico using homology-based searches (Pasquinelli et al., 2000; Lagos-Quintana et al., 2001; Lau et al., 2001; Lee and Ambros, 2001) or computer algorithms (e.g. MiRScan, MiRSeeker) that predict the capability of a candidate miRNA gene to form the stem loop structure of a pri-mRNA (Grad et al., Mol. Cell, 2003; Lim et al., Genes Dev., 2003; Lim et al., Science, 2003; Lai E C et al., Genome Bio._{}} 2003). An online registry provides a searchable database of all published miRNA sequences (The miRNA Registry at the Sanger Institute website; Griffiths-Jones S, Nuc. Acids Res., 2004). Exemplary, natural miRNAs include lin-4, let-7, miR-10, miRR-15, miR-16, miR-168, miR-175, miR-196 and their homologs, as well as other natural miRNAs from humans and certain model organisms including Drosophila melemogaster, Caenorhabditis elegans, zebrafish, Arabidopsis thalania, mouse, and rat as described in International PCT Publication No. WO 03/029459.

Naturally-occurring miRNAs are expressed by endogenous genes in vivo and are processed from a hairpin or stem-loop precursor (pre-miRNA or pri-miRNAs) by Dicer or other RNAses (Lagos-Quintana et al., Science, 2001; Lau et al., Science, 2001; Lee and Ambros, Science, 2001; Lagos-Quintana et al., Curr. Biol., 2002; Mourelatos et al., Genes Dev., 2002; Reinhart et al., Science, 2002; Ambros et al, Curr. Biol., 2003; Brennecke et al., 2003; Lagos-Quintana et al., RNA, 2003; Lim et al., Genes Dev., 2003; Lim et al., Science, 2003). miRNAs can exist transiently in vivo as a double-stranded duplex but only one strand is taken up by the RISC complex to direct gene silencing. Certain miRNAs, e.g. plant miRNAs, have perfect or near-perfect complementarity to their target mRNAs and, hence, direct cleavage of the target mRNAs. Other miRNAs have less than perfect complementarity to their target mRNAs and, hence, direct translational repression of the target mRNAs. The degree of complementarity between an miRNA and its target mRNA is believed to determine its mechanism of action. For example, perfect or near-perfect complementarity between a miRNA and its target mRNA is predictive of a cleavage mechanism (Yekta et al., Science, 2004), whereas less than perfect complementarity is predictive of a translational repression mechanism. In particular embodiments, the miRNA sequence is that of a naturally-occurring miRNA sequence, the aberrant expression or activity of which is correlated with a miRNA disorder.

Naturally-occurring miRNA precursors (pre-miRNA) have a single strand that forms a duplex stem including two portions that are generally complementary, and a loop, that connects the two portions of the stem. In typical pre-miRNAs, the stem includes one or more bulges, e.g. extra nucleotides that create a single nucleotide “loop” in one portion of the stem, and/or one or more unpaired nucleotides that create a gap in the hybridization of the two portions of the stem to each other. Short hairpin RNAs, or engineered RNA precursors, of the invention are artificial constructs based on these naturally occurring pre-miRNAs, but which are engineered to deliver desired RNAi agents (e.g. siRNAs of the invention). By substituting the stem sequences of the pre-miRNA with sequence complementary to the target mRNA, a shRNA is formed. The shRNA is processed by the entire gene silencing pathway of the cell, thereby efficiently mediating RNAi.

MicroRNAs (miRNAs) are small endogenous non-coding RNAs that post-transcriptionally regulate gene expression by binding with imperfect complementarity in 3′ untranslated regions (3′-UTR) of their target messenger RNAs (mRNAs). MiRNAs are 18-25 nucleotide single-stranded small RNAs associated with a complex of proteins which is called RNA-induced silencing complex (RISC)-like ribonucleoprotein particle (miRNP). This complex inhibits translation or, depending on the degree of Watson-Crick complementarity, induces degradation of target mRNAs. These small RNAs are usually generated from non-coding regions of many gene transcripts and function to suppress gene expression by translational repression. MiRNAs have been shown to play important roles in development, cell growth, and differentiation. Recent studies have highlighted the role of miRNAs in various disease states and in regulating host-pathogen interactions. For example, mRNAs have been implicated in cardiovascular disease, inflammation, viral infections, and cancers. Hence, disease-associated miRNAs could become potential targets for therapeutic intervention.

In embodiments where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g. within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g. 2, 3, 4, 5, or 6) may be targeted in certain embodiments.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

As discussed above, in various embodiments antisense oligonucleotides or RNA molecules include oligonucleotides containing modifications. A variety of modifications are known in the art and contemplated for use in the present invention. For example oligonucleotides containing modified backbones or non-natural internucleoside linkages are contemplated. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

The term “RNA Induced Silencing Complex,” and its acronym “RISC,” refers to the set of proteins that complex with single-stranded polynucleotides such as mature miRNA or siRNA, to target nucleic acid molecules (e.g., mRNA) for cleavage, translation attenuation, methylation, and/or other alterations. Known, non-limiting components of RISC include Dicer, R2D2 and the Argonaute family of proteins, as well as strands of siRNAs and miRNAs.

Methods

In one embodiment, the invention provides a method of increasing production of a protein of interest in a cell. The method includes contacting the cell with miRNA of the present disclosure, siRNA of the present disclosure, or both, to increase protein production.

The protein of interest for use with the invention may be any protein which can be expressed in a cell. For example, the protein may be a cytosolic, secreted or membrane protein. The term “polypeptides/protein” is used broadly to refer to macromolecules comprising linear polymers of amino acids which may act in biological systems, for example, as structural components, enzymes, chemical messengers, receptors, ligands, regulators, hormones, and the like. Such polypeptides/proteins would include functional protein complexes, such as antibodies. The term “antibody” is used broadly herein to refer to a polypeptide or a protein complex that can specifically bind an epitope of a polypeptide or antigen. As used in this invention, the term “epitope” refers to an antigenic determinant on a polypeptide or an antigen, such as a cell surface marker or receptor, to which the paratope of an antibody binds.

Generally, an antibody contains at least one antigen binding domain that is formed by an association of a heavy chain variable region domain and a light chain variable region domain, particularly the hypervariable regions. An antibody can be a naturally occurring antibodies, for example, bivalent antibodies, which contain two antigen binding domains formed by first heavy and light chain variable regions and second heavy and light chain variable regions (e.g., an IgG or IgA isotype) or by a first heavy chain variable region and a second heavy chain variable region (V_{HH} antibodies), or on non-naturally occurring antibodies, including, for example, single chain antibodies, chimeric antibodies, bifunctional antibodies, and humanized antibodies, as well as antigen-binding fragments of an antibody, for example, an Fab fragment, an Fd fragment, an Fv fragment, and the like.

Generally, an antibody contains at least one antigen binding domain that is formed by an association of a heavy chain variable region domain and a light chain variable region domain, particularly the hypervariable regions. Antibodies include polyclonal and monoclonal antibodies, chimeric, single chain, and humanized antibodies, as well as Fab fragments, including the products of an Fab or other immunoglobulin expression library. Antibodies which consists essentially of pooled monoclonal antibodies with different epitopic specificities, as well as distinct monoclonal antibody preparations are provided. Monoclonal antibodies are made by methods well known to those skilled in the art. The term antibody as used in this invention is meant to include intact molecules as well as fragments thereof, such as Fab and F(ab′)₂, Fv and SCA fragments which are capable of binding an epitopic determinant on a protein of interest. An Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

Cells for use with the invention generally include eukaryotic cells, such as animal cells. In embodiments, the cells are mammalian cells, such as HEK or CHO cell. However the invention contemplates use of any cell line commonly known in the art for protein production.

miRNAs and siRNAs may be introduced into the cells, according to methods well known in the art. Similarly, the protein of interest may be introduced into the cells, according to methods well known in the art. Typically, a gene encoding the protein is inserted into a plasmid or vector, and the resulting construct is then transfected into appropriate cells, in which the protein is then expressed, and from which the protein is ultimately purified.

In embodiments, a host cell transfected with an expression vector encoding a protein of interest can be cultured under appropriate conditions to allow expression of the protein to occur in the presence of the miRNAs and siRNAs of the invention. The protein may be secreted, by inclusion of a secretion signal sequence, and isolated from a mixture of cells and medium containing the protein. Alternatively, the protein may be retained cytoplasmically and the cells harvested, lysed and the protein isolated. A cell culture includes host cells, media and other byproducts. Suitable media for cell culture are well known in the art. The proteins can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the protein.

In embodiments, an increase in production of the protein greater than that of a control cell not contacted with the miRNA or siRNA is indicative of increased protein production in the cell. In various embodiments, the protein production is increased greater than 1.1, 1.2, 1.3. 1.4, 1.5, 1.6, 1.7, 1.8, 1.9, 2.0 times or more as compared to the control cell not contacted with the miRNA or siRNA.

miRNAs and siRNAs

The invention also provides miRNAs and siRNAs for use in increasing protein production, as well as genome editing methodologies to increase protein production.

In embodiments, the miRNA may be one or more miRNAs including a sequence selected from SEQ ID NOs:1-26, and any combination thereof. For example, the miRNA may be one or more miRNAs selected from SEQ ID NOs:1-4, 20, 21 and 25. In one embodiment, the miRNA includes those as set forth in SEQ ID NOs:2, 3, 20, 21 or 25. In some embodiments, the miRNA includes a common sequence motif as set forth in SEQ ID NO:28 or SEQ ID NO:29. For example, the miRNA has a sequence selected from SEQ ID NOs:4, 16 and 22.

In embodiments, the invention provides an isolated nucleic acid sequence including the miRNA sequence of the invention operably linked to a heterologous promoter. The miRNA sequence may have a length of about 6-25 nucleotides and include a sequence as set forth in SEQ ID NOs:1-26. Similarly, the miRNA sequence may have a length of about 6-25 nucleotides and include a sequence as set forth in SEQ ID NO:28 or 29.

In embodiments, the siRNA is one or more siRNA is that inhibits expression of a gene set forth in Table 3, inhibition of which has been determined to increase protein expression. Such siRNAs include those having a sequence set forth in SEQ ID NOs:38-212.

In some embodiments, the siRNA inhibits one or more genes listed in Table 3, such as one or more of INTS1, INTS2, HNRNPC, CASP8AP2, OAZ1, ODC1, AZIN1, PPP2R1A, PRPF19, CHAF1A, CCT2, EEF1B2, or a combination thereof. In an exemplary embodiment, the siRNA inhibits OAZ1 and has a sequence as set forth in SEQ ID NO:155 or SEQ ID NO:156.

In some embodiments, genome editing tools are used to inhibit or silence one or more genes listed in Table 3, such as one or more of INTS1, INTS2, HNRNPC, CASP8AP2, OAZ1, ODC1, AZIN1, PPP2R1A, PRPF19, CHAF1A, CCT2, EEF1B2, or a combination thereof.

The miRNAs and siRNAs of the present invention may include naturally occurring nucleotides as well as non-naturally occurring nucleotide analogs. Such molecules may also include modified backbones or non-natural internucleoside linkages as discussed herein as well as modifications at the 5′, 3′ or both the 5′ and 3′ terminus.

Kits

The invention also provides a kit for increasing protein production in a cell. The kit includes a miRNA of the present invention, for example, a miRNA sequence having a sequence as set forth in SEQ ID NOs:1-26, and a siRNA which inhibits expression of a gene set forth in Table 3, for example, an siRNAs having a sequence set forth in SEQ ID NOs:94-212.

Screening

In another embodiment, the invention provides a screening method for obtaining miRNAs for enhancing expression of a protein. The method includes: a) contacting a cell comprising a detectably labeled protein with a plurality of miRNAs; and b) measuring protein production prior to and after contacting with the miRNAs, wherein an increase in expression of the protein after contact is indicative of an miRNA for enhancing expression of the protein. In one aspect, the invention provides for assessing the functionality of the enhanced protein produced.

In embodiments, the detectable label is used to detect expression of the labeled protein. Such labels are commonly known in the art and include, for example, luciferase (LUC), β-lactamase, chloramphenicol acetyltransferase (CAT), adenosine deaminase (ADA), aminoglycoside phosphotransferase (neo, G418), dihydrofolate reductase (DHFR), hygromycin-β-phosphotransferase (HPH), thymidine kinase (TK), β-galactosidase (β-gal), and xanthine guanine phophoribosyltransferase (XGPRT), affinity or epitope tags, and fluorescent proteins. In one embodiment the detectable label is green fluorescent protein (GFP) or enhanced green fluorescent protein (eGFP).

ZFN/TALEN/CRISPR

Zinc-finger nucleases (ZFNs), transcription activator-like effector nucleases (TALENs) and CRISPR/Cas9 systems all comprise a powerful class of genome editing tools that are redefining the boundaries of biological research.

These chimeric nucleases are composed of programmable, sequence-specific DNA-binding modules linked to a nonspecific DNA cleavage domain. ZFNs and TALENs enable a broad range of genetic modifications by inducing DNA double-strand breaks that stimulate error-prone non-homologous end joining or homology-directed repair at specific genomic locations. There are potential therapeutic applications of ZFNs and TALENs.

CRISPR/Cas-based RNA-guided DNA endonucleases are the newest of the genome editing tools, and very powerful.

The following example is provided to further illustrate the advantages and features of the present invention, but are not intended to limit the scope of the invention. While they are typical of those that might be used, other procedures, methodologies, or techniques known to those skilled in the art may alternatively be used.

### Example 1

### Improved Protein Production Using miRNAs

This Example sets forth a high-throughput screening strategy for identifying miRNAs that can improve functional expression of the model membrane protein-Neurotensin Receptor type 1 (NTSR1). Belonging to the G protein-coupled receptors (GPCRs) superfamily and interacting with its ligand neurotensin, NTSR1 plays important roles in Parkinson's disease, pathogenesis of schizophrenia, modulation of dopamine neurotransmission, hypothermia, and antinociception and in promoting growth of cancer cells. The structure of a stabilized NTSR1 mutant with T4 lysozyme replacing most of the third intracellular loop was recently determined. Previously, the inventors constructed inducible suspension mammalian HEK293 cells expressing functional NTSR1, that allowed one to obtain 1 mg purified receptor per liter of cell culture with a viable cell density of 1.4 million cells/mL. Here the ability to improve receptor expression by applying the powerful miRNA tool is explored. This study describes the implementation of high-throughput image-based screen with NTSR1-GFP-expressing cells using a human miRNA mimic library comprising 875 miRNA mimics.

Materials and Methods

Construction of Expression Plasmid pJMA-NTSR1-GFP

Truncated wild type NTSR1 (T43-K396) was subcloned into the tetracycline inducible plasmid pJMA111 replacing the serotonin transporter construct using KpnI and NotI restriction sites. Thus NTSR1 was placed downstream of the tetracycline-controlled CMV promoter and had an eGFP-deca-histidine tag fused to its C-terminal (FIG. 7).

Construction of Stable NTSR1-GFP-Expressing T-REx-293 Cell Line

The T-REx-293 cell line was maintained as an adherent culture in DMEM containing 10% certified FBS and 5 μg/mL blasticidin (Invitrogen). The cells were transfected with the plasmid pJMA-NTSR1-GFP using Lipofectamine™ 2000 according to the manufacturer's protocol (Life Technologies). One day after transfection, cells were transferred into fresh DMEM medium containing 200 μg/mL zeocin (Invitrogen) and the medium was replaced every three days. Two weeks later, ten cell clones were separately expanded into two T-flasks each. Cells in one T-flask were harvested during the exponential growth phase and frozen in 10% DMSO for storage. Cells in the other T-flask were induced with 1 μg/mL tetracycline for 24 hrs, after reaching 80% confluency. Cells were then detached from the flask and washed with cold PBS. After adjusting the cell density to ˜1×10⁶ cells per mL, protease inhibitors (Roche) were added and the cell suspension was frozen on dry ice in 1 mL aliquots. NTSR1 expression levels were determined by [³H]NT binding and the clone with the highest expression level was selected for further experiments. The selected stable T-REx-293-NTSR1-GFP high expressor was then routinely maintained in DMEM containing 10% certified FBS, 5 μg/mL blasticidin and 200 μg/mL zeocin.

High-Throughput miRNA Screen

T-REx-293-NTSR1-GFP cells were screened with a miRNA mimic library (Qiagen) based on Sanger miRBase™ 13.0 and consisting of 875 miRNAs mimics. For transfection, 0.8 pmol of each mimic was spotted to 384 well plate wells (Corning) and 20 μL of serum-free DMEM containing 0.1 μL of Lipofectamine™ RNAiMax (Life Technologies) was then added to each well. This lipid-miRNA mixture was incubated at ambient temperature for 30 min prior to adding 2000 cells in 20 μL of DMEM containing 20% certified FBS (Gibco). Transfected cells were incubated at 37° C. in 5% CO₂ for 72 hours and induced with 1 μg/mL tetracycline for 24 hours for NTSR1-GFP expression. Cells were then fixed with 2% paraformaldehyde (Electron Microscopy Sciences), stained with Hoechst™ 33342 (Life Technologies) for 45 minutes and gently washed with PBS. Plates were imaged with an ImageXpress Micro XL™ (Molecular Devices). Total cell number and per cell green fluorescence intensity were calculated using MetaXpress™ software (Molecular Devices) employing the Multi-Wavelength Cell Scoring™ application module. All screening plates had a full column (16 wells) of SilencerSelect™ Negative Control #2 (Life Technologies) and the median value of each plate's negative control column was used to normalize corresponding sample wells. A full column of positive control siRNA targeting GFP (GFP-22 siRNA, Qiagen) was also used as on-plate reference for transfection efficiency. The median absolute deviation (MAD)-based z-score was calculated for each sample.

Validation Transfection

Validation transfections were performed in 12-well plates with miScript™ miRNA mimics (Qiagen, Cat. No. 219600-S0), SilencerSelect™ Negative Control #2 and lethal control siRNA (Qiagen AllStars Cell Death Control™) served as a control for transfection efficiency. Cells were transfected as described for screening except 0.15 million cells were transfected with 40 nM miRNA using 6.25 ul Lipofectamine™ RNAiMax in a total volume of 1 mL of media. 72 hours after transfection, cells were induced with 1 μg/mL tetracycline. 24 hours later, cells from each well were detached with non-enzymatic cell dissociation buffer (Gibco, Cat. No. 13150-016) and washed twice with cold PBS. Cell densities and viability were determined by trypan blue exclusion using a CEDEX™ cell quantification system (Roche, Mannheim, Germany). Based on the counts, cell densities were adjusted to 0.5 million cells/ml with PBS and then subject to flow cytometry analysis. The remaining cells were pelleted and frozen on dry ice for [³H]NT binding assays.

Flow Cytometry Analysis

Cells harvested from validation transfection step were diluted to 0.2 million cells/ml with cold PBS for flow cytometry analysis. Green fluorescence was measured with Guava Easycyte 5HT and Incyte software (Millipore). The green fluorescence signal and cell gating were adjusted using uninduced T-REx-293-NTSR1-GFP cells, with more than 99% of the cells in low fluorescence range (<100). The setting was kept same for acquisition of all cell samples.

Analytical Solubilization of NTSR1

The detergents n-Dodecyl-β-D-maltoside (LM), 3-[(3-cholamidopyropyl) dimethylammonio]-1-propane sulfonate (CHAPS) and cholesteryl hemisuccinate Tris salt (CHS) were obtained from Anatrace. Cell pellets from 2 mL of suspension culture were suspended in Tris-glycerol-NaCl buffer. Then the detergents LM, CHAPS and CHS were added to give a final buffer composition of 50 mM TrisHCl pH 7.4, 200 mM NaCl, 30% (v/v) glycerol, 1% (w/v) LM, and 0.6% (w/v) CHAPS and 0.12% (w/v) CHS in a total volume of 0.5 mL. The samples were placed on a rotating mixer at 4° C. for 1 hour. Cell debris and non-solubilized material were removed by ultracentrifugation (TL100 rotor, 60 k rpm, 4° C., 30 min in Optima Max™ bench-top ultracentrifuge, Beckman), and the supernatants containing detergent-solubilized NTSR1 were used to determine the total number of expressed receptors by a detergent-based radio-ligand binding assay (see below).

Ligand Binding Assay

Tritiated neurotensin agonist [³H]NT ([3,11-tyrosyl-3,5-³H(N)]-pyroGlu-Leu-Tyr-Glu-Asn-Lys-Pro-Arg-Arg-Pro-Tyr-Ile-Leu; SEQ ID NO:27) was purchased from Perkin Elmer. Ligand-binding assays with detergent-solubilized receptors were carried out in TEBB assay buffer (50 mM Tris pH 7.4, 1 mM EDTA, 40 μg/mL bacitracin, 0.1% BSA) containing 0.1% (w/v) LM, 0.2% (w/v) CHAPS and 0.04% (w/v) CHS. For one-point assays, receptors were incubated with 2 nM [³H]NT on ice for 1 hour in a volume of 150 μL. The concentration of [³H]NT used was at least 5-fold above the apparent dissociation constants for detergent-solubilized NTSR1 to allow high receptor occupancy. Separation of the receptor-ligand complex from free ligand (100 μL) was achieved by centrifugation-assisted gel filtration using Bio-Spin™ 30 Tris columns (BioRad), equilibrated with RDB buffer (50 mM Tris-HCl, pH 7.4, 1 mM EDTA, 0.1% (w/v) LM, 0.2% (w/v) CHAPS, 0.04% (w/v) CHS). Non-specific [³H]NT binding of 220 dpm was subtracted from total binding to calculate the total amount of receptors in T-REx-293-NTSR1-GFP cells. The number of functional NTSR1 was estimated by specific [³H]NT binding assuming one ligand-binding site per receptor molecule. The number of cells in the assay was derived by cell counting at cell harvest. This approach led to the calculation of the parameter “receptors/cell”.

Validation with Luciferase-Expressing Cells

CMV-Luc2-HygroHEK293 cell line constitutively expressing luciferase is purchased from Promega. Validation transfections were performed in 12-well plates with miScript miRNA mimics (Qiagen, Cat. No. 219600-S0), SilencerSelect™ Negative Control #2 and lethal control siRNA (Qiagen AllStars Cell Death Control™) served as a control for transfection efficiency. Cells were transfected in duplicates as described above in the screening method with the following modification: 0.1 million cells were transfected with 40 nM miRNA using 6.25 ul Lipofectamine™ RNAiMax in a total volume of 1 mL of media. 72 hours after transfection, 500 μL of ONE-Glo™ Reagent (Promega) was added to one set of replicates for luciferase activity quantification and 500 μL of CellTiter-Glo™ Reagent (Promega) was added to the second set of replicates for viable cell density measurement. All plates were incubated at room temperature for 20 minutes to stabilize luminescent signal and then measured with SpectraMax i3™ plate reader (Molecular Devices). Per cell luciferase production was calculated from overall luciferase activity and viable cell number.

Validation with GPC3-hFc-Expressing Cells

HEK-GPC3-hFc cell line constitutively secreting glypican-3 hFc-fusion protein (GPC3-hFc) was a gift from the National Cancer Institute of the NIH. Cells were grown in DMEM supplemented with 10% FBS in a humidified incubator set at 37° C. and 5% CO₂.

Cells were transfected in 12-well plates as described above in the screening method with the following modification: 0.15 million cells were transfected with 40 nM miRNA using 6.25 ul Lipofectamine™ RNAiMax in a total volume of 1 mL of media. 6 days after transfection, cell culture supernatant was collected and cleared using centrifuge for GPC3-hFc concentration determination with ELISA and cells were detached and counted by trypan blue exclusion using a CEDEX™ cell quantification system (Roche, Mannheim, Germany). Per cell GPC3-hFc production can be calculated from overall GPC3-hFc yield and viable cell number.

ELISA for GPC3-hFc Concentration Determination

AffiniPure™ F(ab′)₂ Fragment Goat Anti-Human IgG (min X Bov, Ms, Rb Sr Prot, Cat. 109-006-170, Jackson Immunology) was used to coat a 96-well plate at 5 μg/mL in PBS buffer, 50 μL per well, at 4° C. overnight. After the plate was blocked with 2% BSA in PBS buffer, pre-diluted cell culture supernatant was added, and the plate was incubated at room temperature for one hour to allow binding to occur. After the plate was washed twice with PBS containing 0.05% Tween 20, Peroxidase-conjugated AffiniPure™ Goat-anti-uman IgG (Cat. 109-035-098, Jackson Immunology) was added at 1:4000 dilutions, 50 ul/well. Following incubating at room temperature for one hour, the plate was washed 4 times and detected with Peroxidase Substrate System (KPL).

Figure Legends

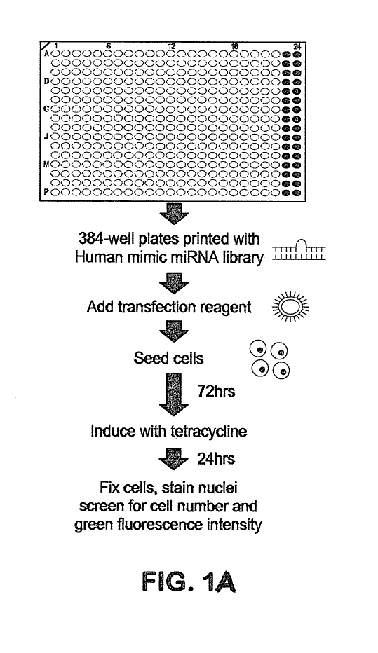

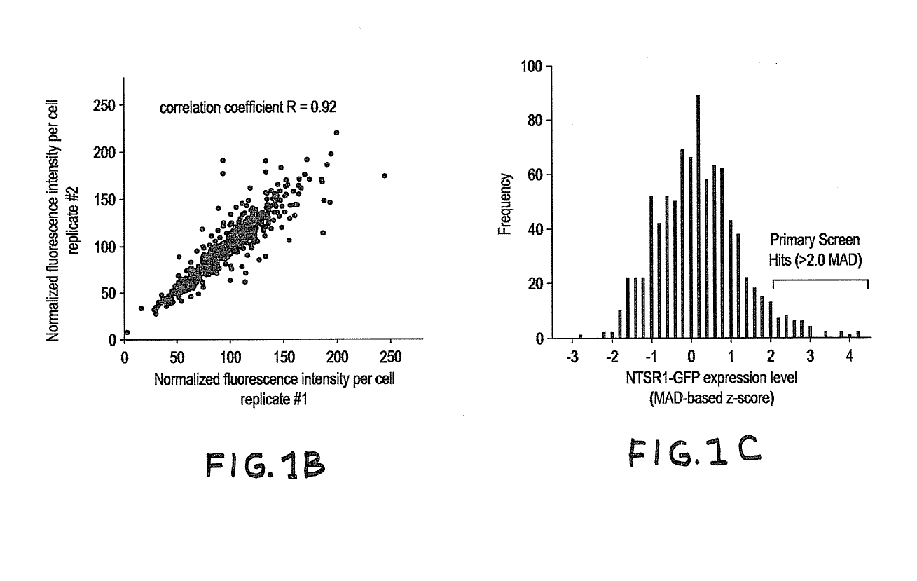

FIG. 1: miRNA screen with stable T-REx-293-NTSR1-GFP cell line. (A) Workflow of the screen. 72 hours post-transfection with human mimic miRNA library (875 miRNAs) in 384-well format, cells were induced with tetracycline, with fixation and analysis 24 hours later. (B) Correlation plot of replicates from the miRNA library screen. The correction coefficient is 0.92. (C) Distribution of miRNA mimics activity on improved NTSR1 expression; top hits (passing 2.0 MAD thresholds) are highlighted.

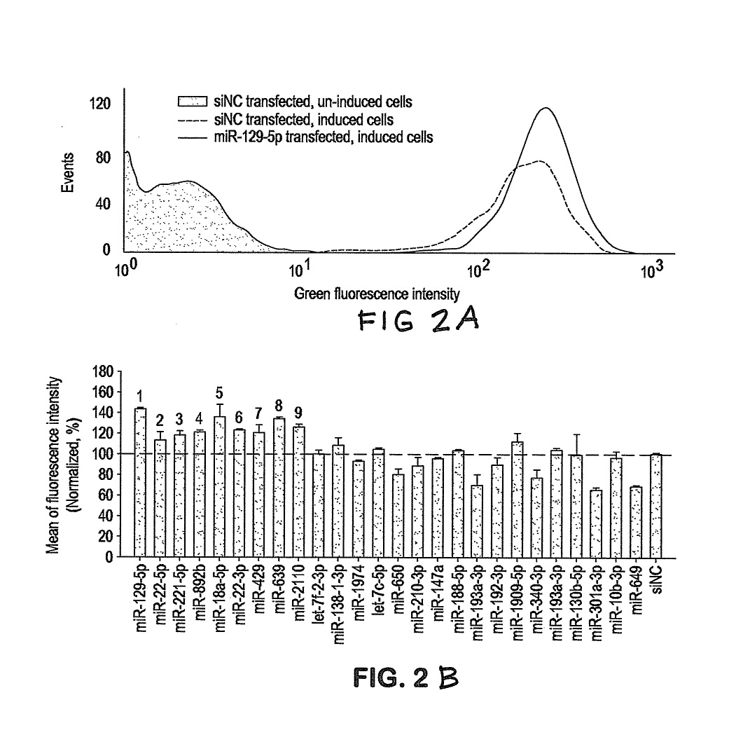

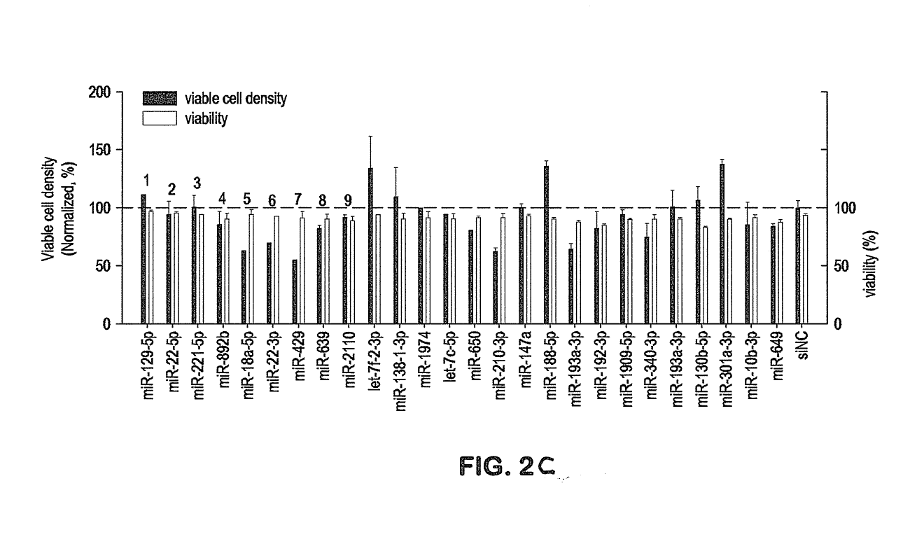

FIG. 2: Flow cytometry analysis on T-REx-293-NTSR1-GFP cells transfected with 26 miRNAs selected from those scoring >2 MAD. (A) Fluorescence histogram of uninduced cells (grey), induced cells transfected with negative control siRNA siN.C. (dash line) and induced cells transfected with miR-129-5p (solid line). Transient transfection of miR-129-5p caused an increase in fluorescence intensity as shown by a right shift compared to control. (B) Testing of the 26 miRNA screen hits by flow cytometry analysis. Cells were transiently transfected with the indicated miRNAs in 12-well plate format and induced with tetracycline. MOF from each sample was normalized to the negative control (siN.C.). Three biological samples were collected for each transfection experiment. Top 9 miRNAs are indicated. (C) Normalized viable cell density and viability of cells transfected with 26 miRNA hits. Error bars represent SEM (standard error of the mean).

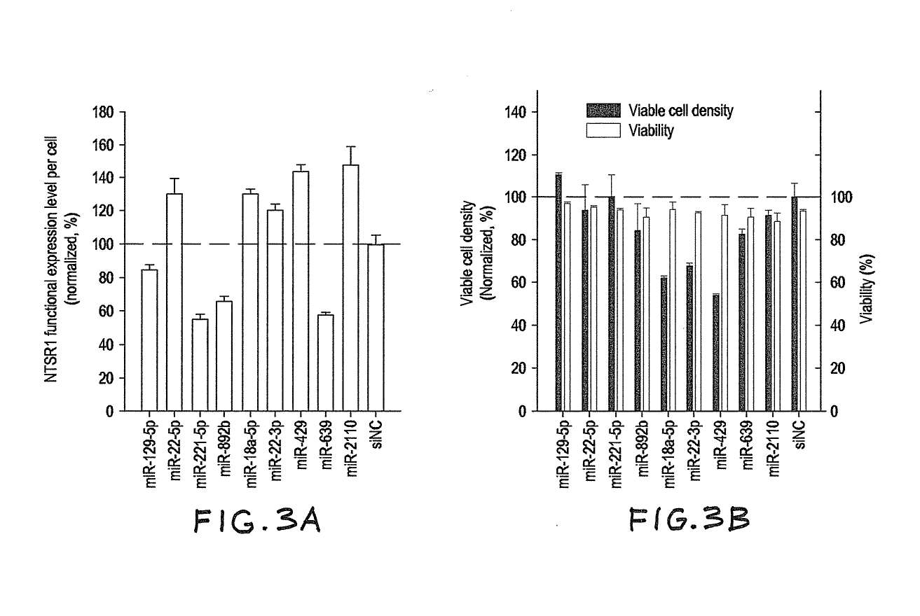

FIG. 3: Validation of improved functional expression of NTSR1 with [³H]NT binding assay. (A) Functional NTSR1 numbers were determined by [³H]NT binding assays using detergent solubilized cells. (B) Cells were counted at harvest and normalized to the control (siN.C.). Two independent experiments were carried out with different passages of T-REx-293-NTSR1-GFP cells, and each independent experiment was tested in duplicate. Error bars indicate SEM.

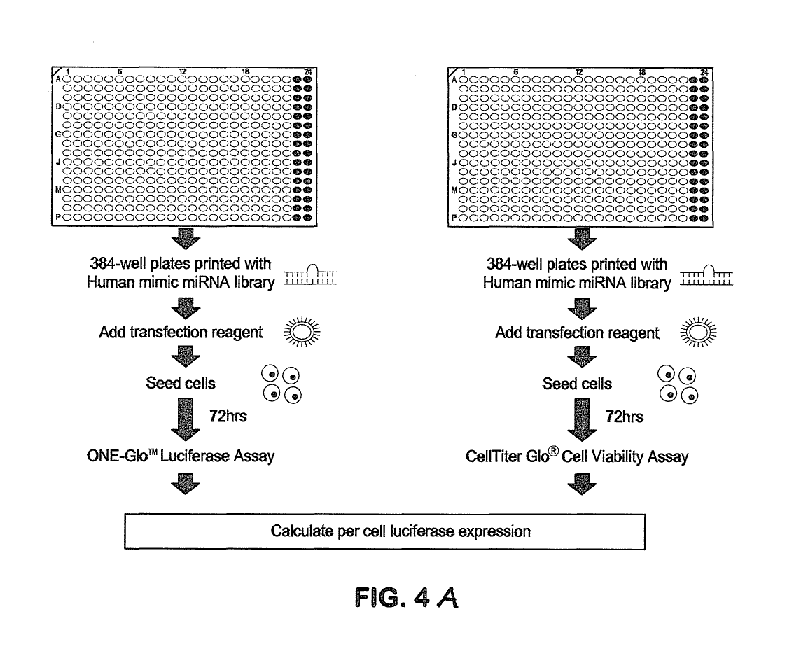

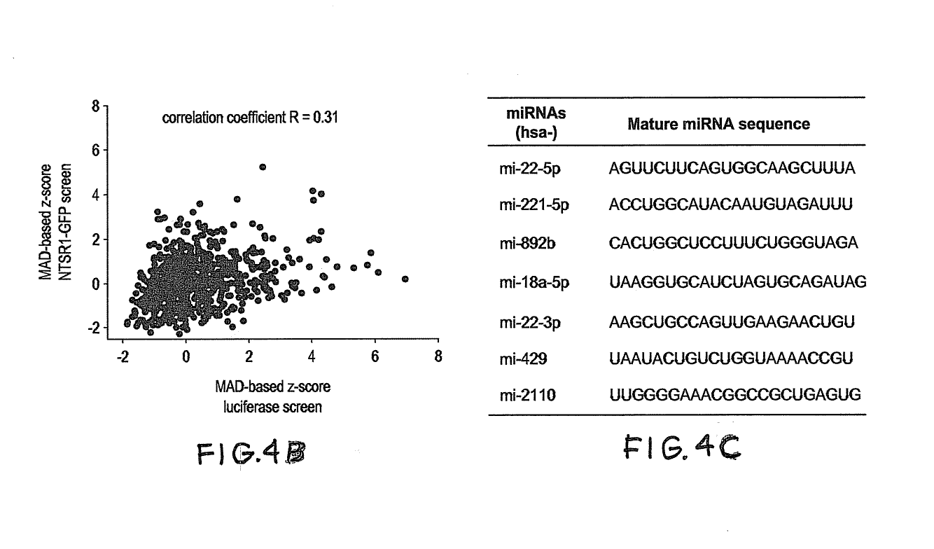

FIG. 4: miRNA screen with stable HEK-CMV-Luc2-Hygro cell line. (A) Workflow of the screen. Transfection with the human miRNA mimic library was performed in duplicate in 384-well format. 72 hours post transfection, one replicate was used for luciferase measurement and the other one was subject to cell viability assay. Data from the two sets of plates were used to calculate per cell luciferase expression level. (B) Correlation plot of screen result from luciferase screen and NTRS1-GFP screen. (C) Top common hits from miRNA library screen with NTSR1 and luciferase as target protein.

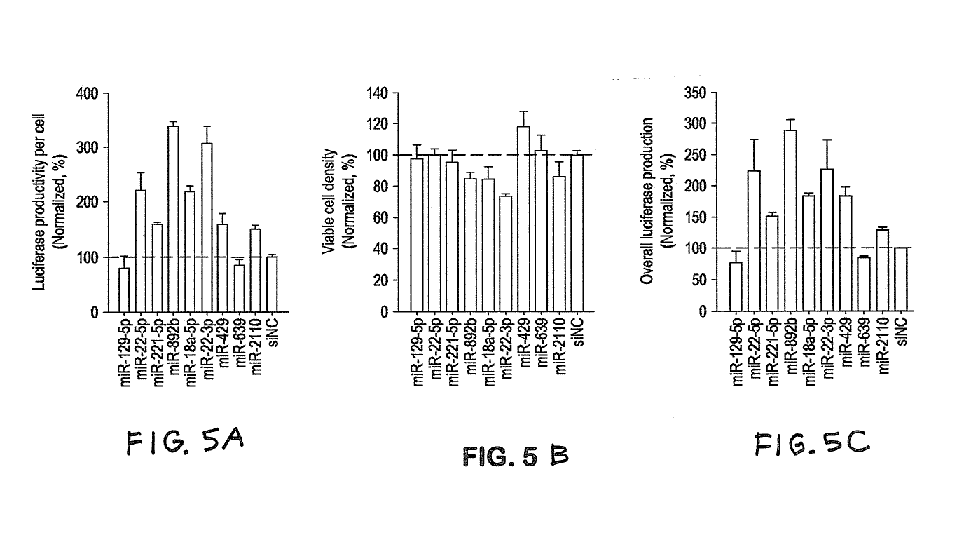

FIG. 5: Validation of improved luciferase activity. CMV-Luc2-Hygro cells were transfected in 12-well plates with the top 9 miRNAs in duplicate. 72 hours post transfection, one replicate was used for luciferase measurement and the other one was subject to cell counting. The experiment was performed twice with different passages of cells. (A) Per cell luciferase activity was determined by ONE-Glo luciferase assay and viable cell density. (B) Viable cell density and (C) Overall luciferase production were normalized to the negative control (siN.C.). For each biological sample, the measurement was done in duplicates. Error bars indicate SEM.

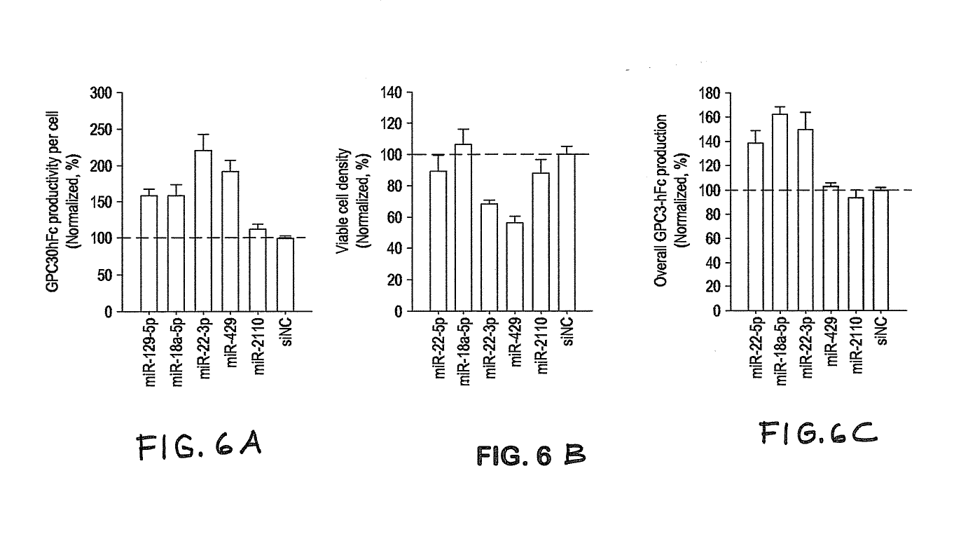

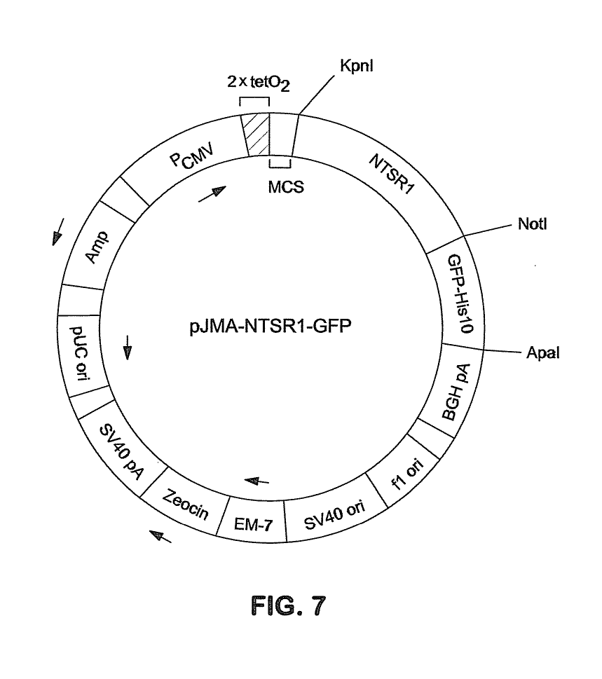

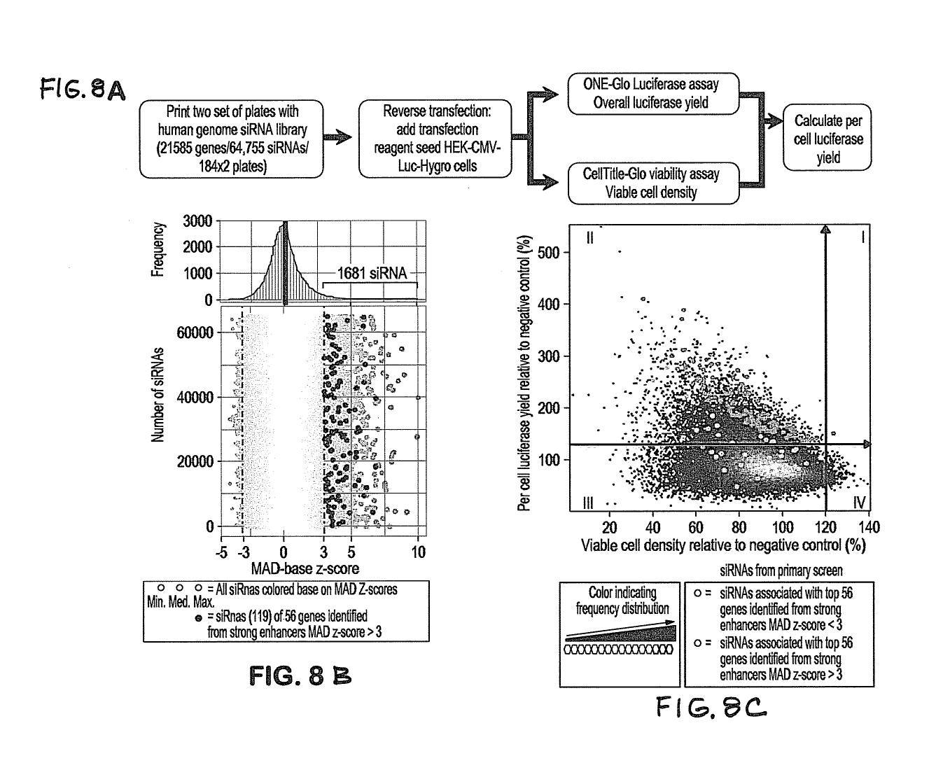

FIG. 6: Improved glypican-3(GPC3) hFc-fusion protein secretion by the five top miRNAs. (A) Per cell GPC3-hFc secretion was determined by ELISA and viable cell density. (B) Viable cell density. (C) Overall GPC3-hFc production were normalized to the negative control (siN.C.) The experiment was performed twice with different passages of cells. For each biological sample, the measurement was done in triplicates. Error bars indicate SEM.

FIG. 7: Plasmid map for pJMA-NTSR1-GFP.

Results

Construction of Inducible T-REx-293-NTSR1-GFP Cell Line for Image-Based Screen

A stable cell line expressing functional wild type NTSR1-GFP fusion was constructed using the inducible T-REx system by transfecting T-REx-293 cells with the pJMA-NTSR1-GFP plasmid (FIG. 7). Ten clones were isolated and their neurotensin receptor expression level upon tetracycline induction was measured by [³H]NT binding assay (data not shown). A high-expressing clone producing 8.4 million receptor molecules per cell was selected for further experiments. The receptors for this clone are located mostly on the plasma membrane as expected.

High-Throughput miRNA Screen for Enhanced NTSR1-GFP Expression

To identify miRNAs that improve NTSR1 expression in T-REx-293-NTSR1-GFP cells, the cells were screened with a library comprised of 875 human miRNA mimics. Cells were transiently transfected with mimics in 384-well format for 72 hours followed by tetracycline-induced expression of NTSR1-GFP fusion protein (FIG. 1A). Twenty four hours after induction, the cells were fixed followed by nuclear staining. Each well was then imaged to obtain total cell number and per cell mean green fluorescent intensity (data not shown). Sample values were normalized based on the median value of each plate's negative control column. A column of positive control siRNA capable of silencing gfp gene was also used as on-plate control for transfection efficiency. GFP-directed siRNA consistently provided a >80% decrease in green fluorescence intensity. To assess reproducibility, the screen was performed in duplicate, resulting in a correlation coefficient of 0.92 (FIG. 1B). Furthermore, the screen was completed again in replicate using cells from a different passage. The correlation between the two independent screens was 0.73. The median absolute deviation (MAD)-based z-score was calculated for each sample, and the distribution of miRNA activity is plotted in FIG. 1C. 40 miRNAs were shown to significantly increase NTSR1-GFP productivity (by passing the 2.0 MAD thresholds. Table 1) in both biological replicates and 26 of them (two thirds of total 40) were selected for follow up analysis. All screen data for the four replicates can be found in Table 1.

TABLE 1
Top hits from human miRNA mimics
screen based on per cell green
fluorescence intensity (MAD > 2.0).

| | | | | Signal | |
|---|---|---|---|---|---|
| | | | MAD- | relative | SEQ |
| Human miR | | | based | to negative | ID |
| ID (hsa-) | Variant | Mature miRNA sequence | z-score | control (%) | NO: |
| miR-221 | 5p | ACCUGGCAUACAAUGUAGAUUU | 5.3 | 248 | 1 |
| miR-429 | - | UAAUACUGUCUGGUAAAACCGU | 4.2 | 212 | 2 |
| miR-22 | 5p | AGUUCUUCAGUGGCAAGCUUUA | 4.0 | 215 | 3 |
| miR-892b | - | CACUGGCUCCUUUCUGGGUAGA | 3.7 | 201 | 4 |
| miR-1974 | - | UGGUUGUAGUCCGUGCGAGAAUA | 3.6 | 201 | 5 |
| miR-210 | 3p | CUGUGCGUGUGACAGCGGCUGA | 3.2 | 183 | 6 |
| let-7f-2 | 3p | CUAUACAGUCUACUGUCUUUCC | 3.0 | 178 | 7 |
| miR-130b | 5p | ACUCUUUCCCUGUUGCACUAC | 2.9 | 178 | 8 |
| miR-188 | 5p | CAUCCCUUGCAUGGUGGAGGG | 2.9 | 177 | 9 |
| miR-301a | 3p | CAGUGCAAUAGUAUUGUCAAAGC | 2.9 | 176 | 10 |
| miR-129 | 5p | CUUUUUGCGGUCUGGGCUUGC | 2.7 | 172 | 11 |
| miR-147a | - | GUGUGUGGAAAUGCUUCUGC | 2.6 | 168 | 12 |
| let-7c | 5p | UGAGGUAGUAGGUUGUAUGGUU | 2.6 | 168 | 13 |
| miR-1909 | 5p | UGAGUGCCGGUGCCUGCCCUG | 2.6 | 169 | 14 |
| miR-138-1 | 3p | GCUACUUCACAACACCAGGGCC | 2.5 | 167 | 15 |
| miR-193b | 3p | AACUGGCCCUCAAAGUCCCGCU | 2.5 | 166 | 16 |
| miR-650 | - | AGGAGGCAGCGCUCUCAGGAC | 2.5 | 163 | 17 |
| miR-639 | - | AUCGCUGCGGUUGCGAGCGCUGU | 2.4 | 165 | 18 |
| miR-10b | 3p | ACAGAUUCGAUUCUAGGGGAAU | 2.4 | 162 | 19 |
| miR-2110 | - | UUGGGGAAACGGCCGCUGAGUG | 2.3 | 160 | 20 |
| miR-22 | 3p | AAGCUGCCAGUUGAAGAACUGU | 2.3 | 158 | 21 |
| miR-193a | 3p | AACUGGCCUACAAAGUCCCAGU | 2.3 | 156 | 22 |
| miR-340 | 3p | UCCGUCUCAGUUACUUUAUAGC | 2.3 | 159 | 23 |
| miR-649 | - | AAACCUGUGUUGUUCAAGAGUC | 2.0 | 150 | 24 |
| miR-18a | 5p | UAAGGUGCAUCUAGUGCAGAUAG | 2.0 | 149 | 25 |
| miR-192 | 3p | CUGCCAAUUCCAUAGGUCACAG | 2.0 | 148 | 26 |

Validation of the Selected miRNA Candidates by Flow Cytometry Analysis

The expression level of NTRS1-GFP following transient transfection of the cells with the top 26 microRNA was measured by flow cytometry (FIG. 2). The un-induced cells exhibited basal GFP expression with only 1% of cells exceeding the background fluorescence (10¹) (FIG. 2A). Following transfection with negative control siRNA (siN.C.) and tetracycline induction, the expression of NTSR1-GFP caused a significant shift in the fluorescence intensity, resulting in a geometric mean of fluorescence (MOF) of 138. A further shift was observed when the cells were transfected with various miRNA mimics followed by tetracycline induction, including miR-129-5p, which led to a MOF of 197. Compared with negative control siRNA, 14 of the 26 miRNAs resulted in an increased MOF. From this group, top 9 miRNAs were selected for further investigation (FIG. 2B). Following the transfection with the 26 selected miRNAs, a large variance was seen in viable cell density (ranged from 54% to 135%, normalized to negative control) but not in viability (ranged from 84% to 97%) (FIG. 2C).

[³H]NT Binding Assay Validation for Improved Functional Expression of NTSR1

The effect of the top 9 miRNAs on the functional expression of NTSR1 was also evaluated by measuring the functional activity of the receptor through the binding of labeled neurotensin ([³H]NT). Although all top 9 miRNAs were shown to improve NTSR1-GFP expression based on GFP fluorescence, only 5 of them (miR-22-5p, miR-18a-5p, miR-22-3p, miR-429 and miR-2110) led to improved functional activity levels of NTSR1 (FIG. 3A). Of these, miR-2110-transfected cells expressed 13.8 million functional neurotensin receptor molecules per cell, which was 48% higher than that from siN.C. In addition, miR-22-5p and miR-22-3p improved functional expression of NTSR1, by 30% and 21% respectively. As seen in FIG. 3B a number of the top 9 miRNAs had negative effect on cell growth and viability.

MiRNA Screen for Enhanced Luciferase Expression

The human mimic miRNA library was also evaluated for its effects on the expression of luciferase in HEK293 cells constitutively expressing luciferase under control of a cytomegalovirus (CMV) promoter. Screening was performed in duplicate in 384-well format. Seventy two hours post-transfection, one set of plates was assayed for luciferase and the other set was used for viable cell density (FIG. 4A). Both luciferase activity and viable cell density were normalized to the median value of each plate's negative control column and the luciferase expression per cell was calculated for each miRNA. Though luciferase and NTSR1 screen exhibited a limited correlation (R=0.31, FIG. 4B), seven out of nine top hits identified from NTSR1 screen (FIG. 4C) also significantly improved per cell luciferase productivity on a per cell basis (passing the >2.0MAD threshold).

Validation of Common Hits

The top 9 miRNAs identified from the NTRS1 screen were examined for their effects on luciferase activity in a 12-well plate format where seven miRNAs improved luciferase activity. (FIG. 5A). MiR-892b and miR-22-3p showed the largest effect on luciferase expression with a 239% and 207% improvement respectively. Although these microRNAs inhibited cell growth (FIG. 5B), the overall production of luciferase from cells transfected with miR-892b and miR-22-3p was still 188% and 127% higher, respectively, than the negative control siN.C. level (FIG. 5C). Interestingly, both miR-22-3p and miR-22-5p showed up as top common hits for NTSR1 and luciferase screen.

Application of Top Common Hits on Secreted Protein

To investigate the impact of top common hits on secreted protein production, the five identified miRNAs (hsa-miR-22-5p, hsa-miR-18a-5p, hsa-miR-22-3p, hsa-miR-429 and hsa-miR-2110) were independently transfected into HEK293 cell line stably expressing secreted hFc-fusion protein: glypican-3 hFc-fusion protein (GPC3-hFc). All five miRNAs enhanced per cell GPC3-hFc secretion (up to 120% improvement, FIG. 6A), while three miRNAs (hsa-miR-22-5p, hsa-miR-18a-5p and hsa-miR-22-3p) effectively enhanced overall GPC3-hFc (up to 62%, FIG. 6C).

Discussion

Integral membrane proteins such as mammalian receptors, ion channels and transporters are vital for medical research. However, obtaining large amounts of functional membrane proteins for medical research, especially structural studies, has been difficult and therefore been a barrier for productive research towards better understanding of their mechanisms and potential medical use. So far, a tetracycline-inducible mammalian expression system has been shown to be an effective method for functional expression of membrane proteins. This inducible system together with optimized production conditions led to a yield of 1 milligram per liter of purified NTSR1. Compared with well-developed prokaryotic hosts such as E. coli, the production of membrane proteins from higher eukaryotic hosts is still in the stage of “trial and error” since engineering tools are limited and the membrane protein synthesis, insertion, folding and trafficking are not completely understood.

To improve the production of these proteins, a bottom-up screening approach using human miRNA mimics library was implemented to identify candidates that lead to improved expression of the GPCR from the T-Rex-293 cells. This approach has previously proven effective for apoptosis screening and recombinant secreted protein screening in CHO cells. In this study, An image-based high-throughput screening method was developed to detect per cell green fluorescence signal, which is applied as a proxy for the number of molecules of NTSR1 protein expressed per cell. In addition to its high reproducibility (0.92 correlation between technical replicates), this method is cost-effective for protein with a fluorescent label, as no secondary reagent is needed for protein quantification. It is also high throughput and high-capacity, as cells are fixed and the screening is not time-sensitive compared to live-cell processes such as flow cytometry. This screen methodology can be applied to other membrane or intracellular protein candidates when the targeted protein is fused with GFP. Although GFP fusion has been widely used for membrane protein overexpression screen and purification in a variety of hosts, it is possible that the N-terminal GFP fusion may mask signal sequence essential for protein insertion. This may compromise folding or correct localization of the desired membrane protein. C-terminal GFP fusion, on the other hand, is preferable as it is generally better in maintaining the localization and function of the native protein with exceptions when C-terminal contains an essential functional segment.

Among the 875 human miRNA mimics tested, 40 mimics consistently led to significant improvement in per cell green fluorescence levels, exhibiting an average MAD-based-z-score higher than 2.0. Among the top 40 candidates, miR-892b, miR193b-3p and miR-193a-3p share the same seed sequence (ACUGGC; SEQ ID NO:28), indicating that they may comprise an overlap in target genes. Similarly, miR-129-3p and miR-129-1-3p also share a same seed sequence (AGCCCU; SEQ ID NO:29).

The activity of two thirds of the 40 mimics was confirmed further by flow cytometry and the 9 mimic candidates contributing to the highest per cell green fluorescence signal were further tested in the [³H]NT binding assay. Five out of the nine mimics showed up to 48% improvement in functional expression of NTSR1. From these five, miR-2110 is a novel miRNA that has been identified but not studied. The other four miRNAs (miR-429, miR-18a-5p, miR-22-5p and miR-22-3p) have been associated with cancer research in which they have exhibited contradicting effects on cell proliferation, cell growth, and protein production depending on the cell type and stage of cell development. For example, miR-429, a member of the miR-200 family, was shown to suppress tumor growth in human osteosarcoma, while in non-small cell lung cancer (NSCLC), the same miRNA is suggested as a potential target for NSCLC due to its promotion of cell proliferation. miR-18a-5p is part of the miR-17-92 precursor sequence cluster, which is also named Oncomir-1. This miR-17-92 cluster was studied in depth regarding its effect on recombinant EpoFc protein production in CHO cells. Although over-expression of the entire cluster decreased productivity while having no effect on cell growth, the over-expression of miR-17 and miR-92 were shown to increase production.

Of the nine miRNAs that enhanced the expression of the NTSRI-GFP fusion protein, four (miR-129-5p, miR-221-5p, miR-892b and miR-639) were not associated with enhanced binding activity of the agonist in the [³H]NT assay. This may be an indication that NTSR1 could be misfolded in these cells following the enhanced expression. Another observation is that eight of the nine top hits (except miR-129-5p) caused a decrease in the viable cell number. One possible reason for this behavior is that overexpression of NTSR1-GFP could be toxic to the cells. Another possibility is that the introduction of a specific miRNA to the cells is associated with a growth arrest, leading to improved protein production. Since multiple pathways and genes can be targeted by one miRNA, it will be worthwhile to examine which specific genes are down-regulated in these cells and to investigate the mechanism that improved NTSR1 functional expression.

In parallel to the analysis of the miRNA effect on the NTSR1 expression, an HEK293 cell line constitutively expressing luciferase under the control of CMV promoter was subjected to a screening of the same miRNA mimics library. This screen showed low degrees of correlation (R=0.31) with the NTSR1-GFP screen. The low correlation may be the result of the difference between biogenesis process of integral membrane proteins and intracellular soluble proteins; the difference between constitutive expression elements and the inducible expression system; and clonal differences between the two HEK293 cell line used. Despite the overall low correlation between the screens, seven out of nine top miRNAs (except miR-129-5p and miR-639) identified from NTSR1-GFP screen, improved luciferase activity from 50% to 239%. All the final five miRNAs (miR-429, miR-18a-5p, miR-22-5p and miR-22-3p and miR-2110) capable of improving NTSR1 functional expression were also relevant for improving luciferase expression.

These five miRNAs affecting both model proteins were expected to have wider application for other types of proteins. Therefore, they were tested with HEK293 cell line constitutively secreting a Fc fusion proteins with medical importance. Interestingly, all of the five miRNAs were effective in enhancing per cell Fc fusion protein secretion. However, the overall Fc fusion protein yield varied from 10% decrease to 62% increase, partially depending on the viable cell number after miRNA transfection.

### Example 2

### Genome-Scale RNA Interference Screen Identifies Key Pathways and Genes for Improving Recombinant Protein Production in Mammalian Cells

For this example, a genome-wide siRNA screen to identify genes that may influence recombinant protein production, using Photinus pyralis (firefly) luciferase as a reporter protein. With a high-throughput format, 21,585 genes were individually silenced with three different siRNAs, in HEK-CMV-Luc2-Hygro cells constitutively expressing firefly luciferase. The viable cell number and the luciferase activity were measured following the screening and the results were incorporated into genome-wide loss-of-function data. Statistical data analyses were conducted, followed by a validation screen where ten target genes (leading to greatest improvement of luciferase production) were confirmed. Among these selected genes, OAZ1 the gene that encodes antizyme 1, an inhibitor to ornithine decarboxylase, was chosen for more detailed studies, since its silencing caused minimal effect on cell viability.

Materials and Methods

Cell Culture

HEK-CMV-Luc2-Hygro cell line constitutively expressing P. pyralis luciferase (Progema) and HEK-GPC3-hFc cell line constitutively secreting glypican-3 hFc-fusion protein (GPC3-hFc) were maintained in DMEM containing 10% fetal bovine serum (FBS). The inducible T-Rex-SERT-GFP cell line and T-Rex-NTSR1-GFP cell line were maintained as an adherent culture in DMEM containing 10% certified FBS, 5 μg/mL blasticidin and 200 μg/mL zeocin (Invitrogen). All cells were maintained in a humidified incubator set at 37° C. and 5% CO₂.

High-Throughput Genome-Wide Screen for Luciferase Expression

The Silencer Select™ Human genome siRNA library (Ambion), which targets 21,585 human genes with 3 siRNAs per gene, was used for screening. Each siRNA is arrayed in an individual well (Corning 3570, 384 well, white, solid bottom plates). The transfection was done in duplicates: 0.8 pmol of each siRNA was spotted to a well of a 384-well plate (Corning) and 20 μL of serum-free DMEM containing 0.07 μL of Lipofectamine™ RNAiMax (Life Technologies) was then added to each well. This lipid-siRNA mixture was incubated at ambient temperature for 30 min prior to addition of 4000 cells in 20 μL of DMEM containing 20% FBS (Gibco). After incubating the transfected cells at 37° C. in 5% CO₂ for 72 hours, 20 μL of ONE-Glo™ Reagent (Promega) was added to one set of replicates for ‘overall luciferase yield’ quantification and 20 μL of CellTiter-Glo™ Reagent (Promega) was added to the second set of replicates for ‘viable cell density’ measurement. All plates were incubated at room temperature for 20 minutes to stabilize the luminescent signal and the signal was then measured with PerkinElmer Envision 2104 Multilabel™ plate reader. All plates had a full column (16 wells) of Silencer Select™ Negative Control #2 (Life Technologies) for data normalization and a full column of siPLK1 (Ambion Silencer Select, cat#s448) was also used as on-plate reference for transfection efficiency and both controls were also used in all validation transfections.

The 56 genes which got targeted by at least two independent siRNAs (out of three) resulting in enhanced luciferase production with MAD-based z-score>3 from the primary screen were subjected to validation screen using 3 additional Silencer™ siRNAs (Ambion) with different sequences from those used in the primary screen. Ten gene candidates were selected based on the criteria that 3 out of 6 siRNAs displayed a MAD-based z-score>3. The transfection and assay processes were-the same as the primary genome-wide screen. Data visualization was performed in R computational environment (available on the World Wide Web at R-project.org/) by using ‘hexbin’ and ‘ggplot2’ packages.

Statistical Analysis of Primary Screen Data

The screen generated end-point data for ‘overall luciferase yield’ and ‘viable cell density’ in each well. For each plate, the median value of the negative control wells was set as 100% and was used to normalize corresponding sample wells. The ‘overall luciferase yield’ and ‘viable cell density’ were exported as % of negative control and the median absolute deviation (MAD)-based z-score was calculated for each sample.

Gene Ontology (GO) Analysis

In order to get the maximum coverage of GO annotation data for 119 selected siRNA's targeting 56 genes, PANTHER classification system (available on the World Wide Web at pantherdb.org/) and AmiGO 2 GO™ browser were used. The construction of a heatmap was accomplished using Partek Genomics Suite™ software, version 6.6.

Validation Transfection

Ten targeted genes were selected and tested in four HEK 293 cell lines expressing different reporter proteins, glycan-3 hFc-fision protein (GPC3-hFc), neurotensin receptor type 1-GFP (NTSR1-GFP) and serotonin transporter-GFP (SERT-GFP), using 1 representative siRNA for each gene. Transfection was performed in 12-well plate format. 500 μL of serum free DMEM media containing siRNA and Lipofectamine™ RNAiMax was incubated in each well for 20 min at ambient temperature and 500 μL DMEM containing 20% FBS and cells was then added for transfection. The final siRNA concentration in each well was 40 nM. Lipofectamine™ RNAiMax volume and cell seeding number in each well have been optimized for each cell line (Table 2).

TABLE 2
Optimized transfection condition (12-well plate format) for
HEK 293 cells expressing different recombinant proteins.

| | Lipofectamine | Cell seeding |
|---|---|---|
| Cell line | RNAiMax ™ (μl) | number (million) |
| HEK- GPC3-hFc | 3.75 | 0.15 |
| T-Rex-NTSRl-GFP | 6.25 | 0.15 |
| T-Rex-SERT-GFP | 5 | 0.15 |
| HEK-CMV-Luc2-Hygro | 3.75 | 0.1 |

ELISA for Determination of GPC3-hFc Production

5 days after transfection, clarified cell culture supernatant was used for determination of GPC3-hFc concentration by ELISA and cells were detached and counted by trypan blue exclusion using a CEDEX™ cell quantification system (Roche, Mannheim, Germany). AffiniPure™ F(ab′)₂ Fragment Goat Anti-Human IgG (min X Bov, Ms, Rb Sr Prot, Cat. 109-006-170, Jackson Immunology, 5 μg/mL in PBS) was used to coat a 96-well plate (50 μL per well) at 4° C. overnight. After blocking the plate with 2% BSA in PBS, 50 μl of pre-diluted cell culture supernatant was added, and the plate was incubated at room temperature for 1 h to allow binding to occur. After the plate was washed twice with PBS containing 0.05% Tween 20, Peroxidase-conjugated AffiniPure™ Goat-anti-human IgG (Cat. 109-035-098, Jackson Immunology) was added at 1:4000 dilution (50 μL/well). Following incubation at room temperature for 1 hr, the plate was washed 4 times and signals were detected with Peroxidase Substrate System (KPL).

Flow Cytometry Analysis for Determination of NTSR1-GFP and SERT-GFP Production

3 days after transfection, cells were induced with 1 μg/mL tetracycline. 24 hours later, cells from each well were detached with non-enzymatic cell dissociation buffer (Gibco, Cat. No. 13150-016) and washed twice with cold PBS. Cell densities were adjusted to 0.5 million cells/ml with PBS and then subjected to flow cytometry analysis. Green fluorescence was measured with Guava Easycyte 5HT™ and Incyte™ software (Millipore). The green fluorescence signal and cell gating were adjusted using uninduced T-REx-293-NTSR1-GFP cells, with more than 99.5% of the cells in low fluorescence range (<100). The setting was kept the same for all cell samples.

OAZ1 Silencing Studies

HEK-CMV-Luc2-Hygro cells in 6 well plates were transfected with Silencer siRNA for oaz1 gene (Catalog number: AM51331, assay ID: 46078). The transfection was done in 6-well plate format: 0.12 nmol of each siRNA and 1.5 mL of serum-free DMEM containing 11.25 μL of Lipofectamine™ RNAiMax (Life Technologies) was then added to each well. This lipid-siRNA mixture was incubated at ambient temperature for 30 min prior to adding 2×10⁵ cells in 1.5 mL of DMEM containing 20% FBS (Gibco). The transfected cells were incubated at 37° C. in 5% CO₂ and were harvested at 24, 48, 72 and 96 hr. Luciferase activity was determined using ONE-Glo™ Reagent (Promega) and aliquots of transfected cells.

Isolation of RNA and Real-Time qRT-PCR

Cells were trypsinized from 6-well plates, washed twice with cold PBS and cell pellets were flash frozen on dry ice and stored at −80° C. until extraction. RNA was extracted using the RNeasy™ kit (Qiagen) and then treated with DNase using TURBO DNA-Free™ Kit (Life Technologies). cDNA was generated from the RNA using the Maxima Frist Strand cDNA Synthesis Kit for qRT-PCR (Thermo Scientific). The real-time qPCR was performed using Fast SYBR™ Green Master Mix (Life Technologies) in 7900 HT Fast Real Time PCR System™ (Applied Biosystems). The 2^{-ΔΔct} method was used for relative expression analysis with GAPDH as the reference gene. Cells transfected with negative control siRNA and harvested at 24 hr were set as calibrator. Primers used for each gene are:

luc (Promega),
(SEQ ID NO: 30)
5′-TCACGAAGGTGTACATGCTTTGG-3′
and
(SEQ ID NO: 31)
5′-GATCCTCAACGTGCAAAAGAAGC-3′;
ODC1,
(SEQ ID NO: 32)
5′-TAAAGGAACAGACGGGCTCT-3′
and
(SEQ ID NO: 33)
5′-CCATAGACGCCATCATTCAC-3′;
OAZ1:
(SEQ ID NO: 34)
5′-GGAACCGTAGACTCGCTCAT-3′
and
(SEQ ID NO: 35)
5′-TCGGAGTGAGCGTTTATTTG-3′;
GAPDH:
(SEQ ID NO: 36)
5′-CATCAATGGAAATCCCATCA-3′
and
(SEQ ID NO: 37)
5′-TTCTCCATGGTGGTGAAGAC-3′.

Western Blotting

Transfected cells were lysed in buffer containing 50 mM Tris-HCl, pH 7.4, 5 mM EDTA, 150 mM NaCl, 1% Nonidet P-40, and protease inhibitor mixture. Proteins (˜20 μg) were separated by SDS-PAGE (4-12% gel) in MES buffer and transferred to 0.2-μm nitrocellulose membrane for immunodetection using mouse anti-ODC (Sigma, catalog number 01136) and mouse anti-β-actin (BD biosciences, catalogue number 612657) primary antibodies and HRP conjugated anti-mouse secondary antibodies (abCAM, catalog number ab20043). Signals were detected with an ECL Plus chemiluminescence reagent.

Measurement of Cellular Polyamine Concentration

Cells in six-well plates were washed with PBS twice, harvested, and precipitated with 0.1 mL cold 10% trichloroacetic acid (TCA). A total of 50 μL of the TCA supernatant was used for polyamine analysis by an ion exchange chromatographic system (Biochrom). TCA precipitates were dissolved in 0.1 N NaOH and aliquots were used for protein determination by the Bradford method. Polyamine contents were estimated as nmol/mg protein.

Figure Legends

FIG. 8: Genome-wide human siRNA library screen with HEK-CMV-luc2-Hygro cell line. (A) Workflow of the primary screen; (B) Distribution of siRNA effect on improved overall luciperase expression, The 119 siRNAs corresponding to 56 identified genes with strong enhancer MAD z-score (>3) are indicated as black circles. (C) Relative per cell luciferase yield as a function of the relative viable cell viability for each sRNA tested. The 20% increase cutoffs are highlighted and divide the entire population into quadrants (I, II, III and IV). siRNAs associated with top 56 genes >3 are indicated as red circles and those with MAD-z-score <3 as orange circles.

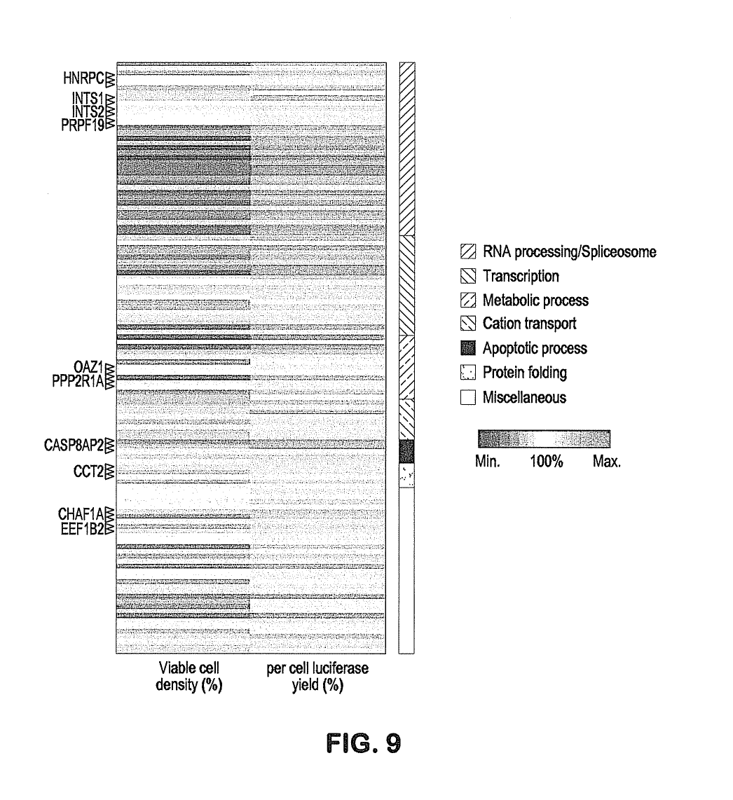

FIG. 9: Functional categorization of strong enhancer siRNA-associated genes. Heat map was generated based on percent viable cell density and per cell luciferase yield for each of the 119 siRNAs that significantly enhanced luciferase production. The values are indicated by range of red (maximum) and blue (minimum) intensities. The functional categories are indicated by bars of different colors and the numbers of siRNAs in each group indicated by the bar lengths.

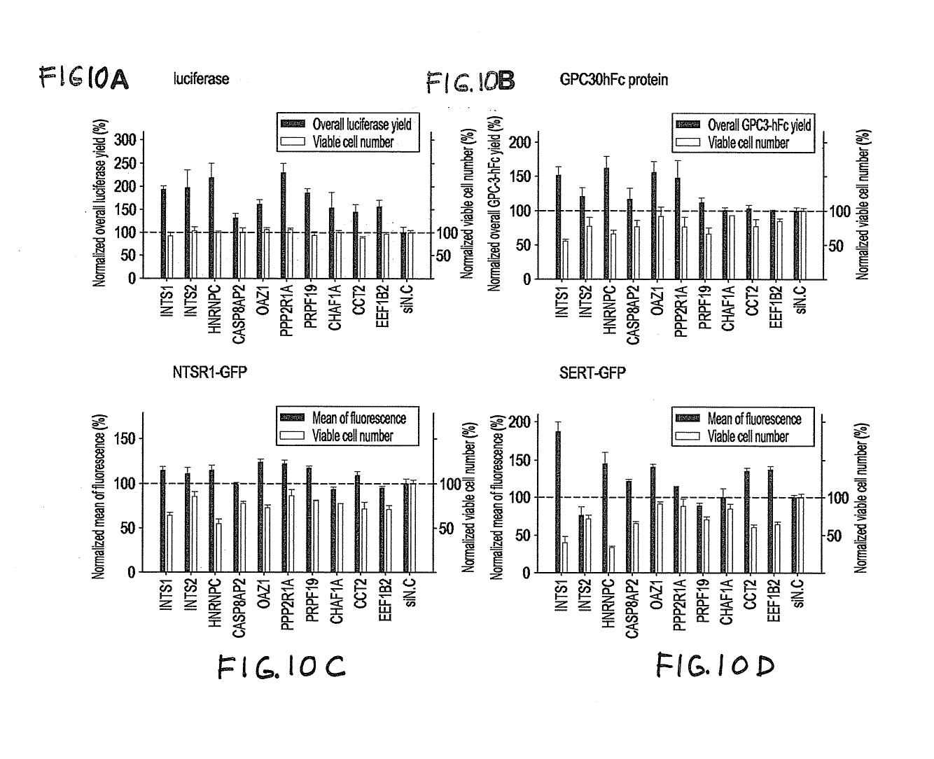

FIG. 10: Effects of the 10 selected enhancer siRNAs on four HEK cell lines expressing different recombinant proteins. (A) Luciferase, (B) GPC3-hFc, (C) NTSR1-GFP, (D) SERT-GFP; Protein expression and cell viability were normalized against cells transfected with the negative control siRNA (siN.C.). The experiment was performed twice with different passages of cells. For each biological sample, the measurement was done in duplicates. Error bars indicate SEM.

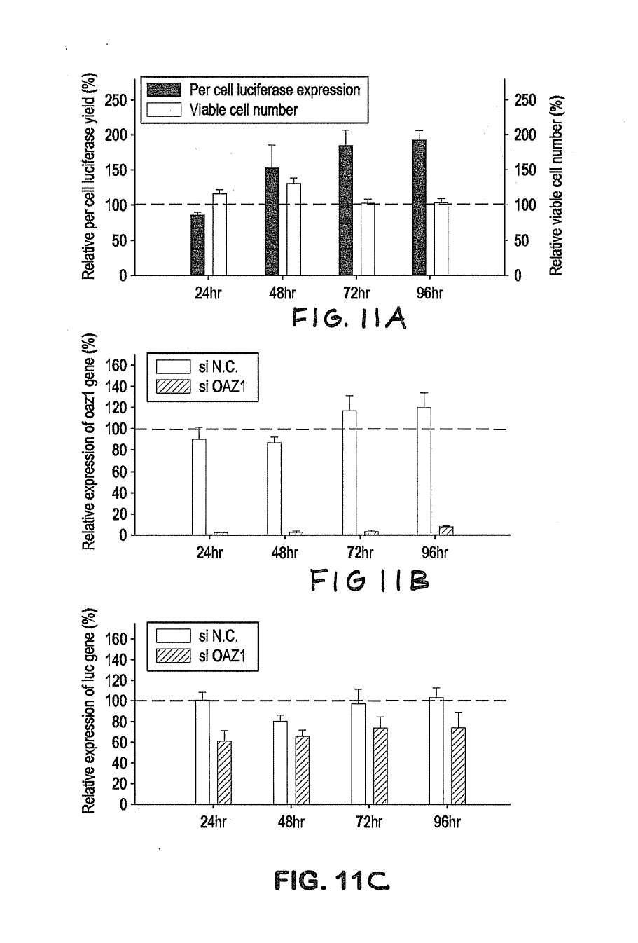

FIG. 11: Time course of the effects of OAZ1siRNA transfection on cell viability and luciferase yield, and the mRNA levels of OAZ1 and luciferase. (A) Cell viability and luciferase protein expression; (B) Relative expression of OAZ1 mRNA; (C) Relative expression of luciferase mRNA. The relative levels in the OAZ1 siRNA-transfected cells were compared to those of cells transfected with negative control siRNA (siN.C.). Transfection was done with two different passages of cells and each biological sample was measured in triplicates. Error bars represent SEM.

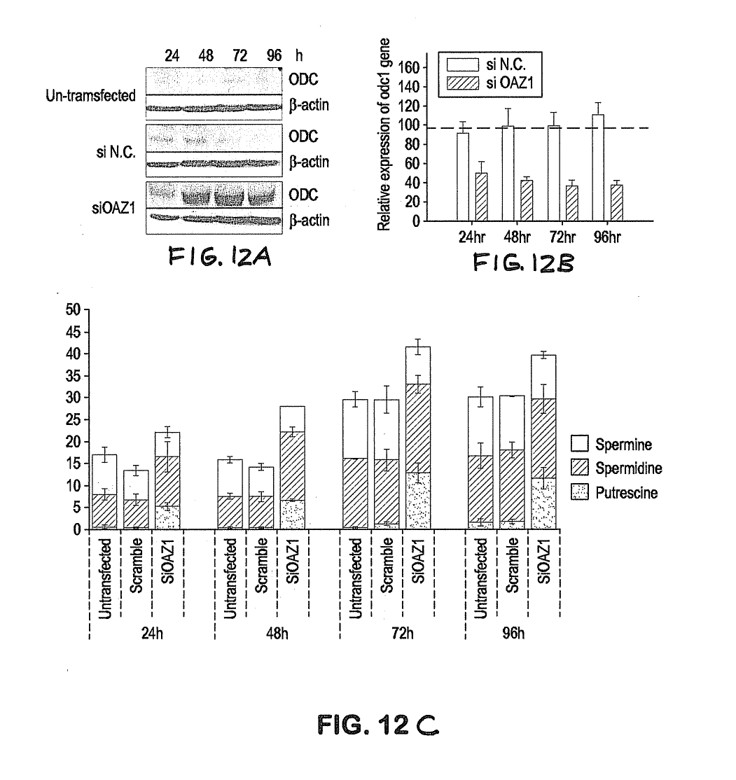

FIG. 12: Time course of the effects of OAZ1 silencing on the levels of ODC protein, ODC mRNA and cellular polyamines. (A) Western blot of ODC, (B) ODC mRNA level, (C) Cellular polyamines concentration. Polyamine concentrations were normalized against total protein and presented as nmol/mg total protein. Transfection was done with two different passages of cells and each biological sample was measured in triplicates. Error bars represent SEM. si N.C. indicates control scramble siRNA.

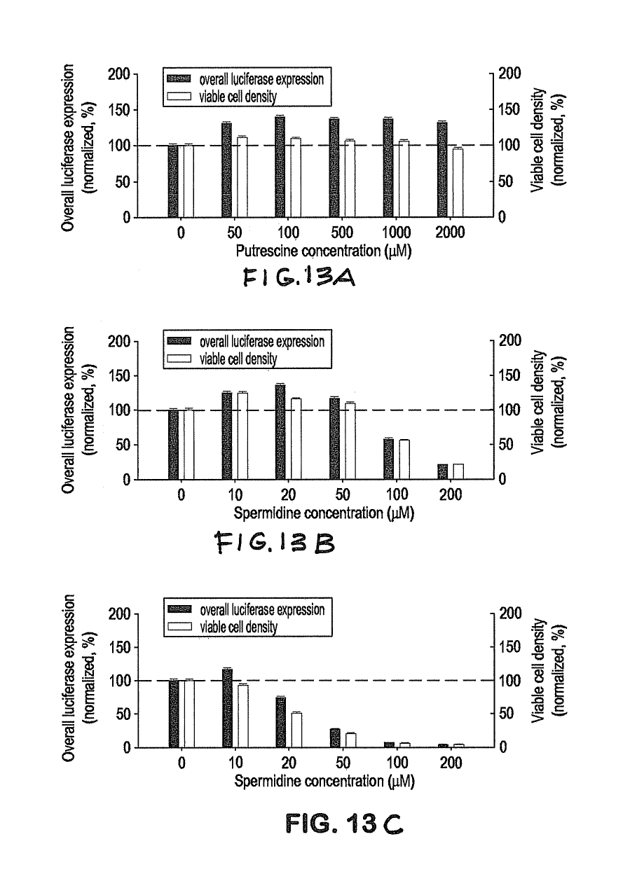

FIG. 13: Effect of exogenous polyamines on luciferase expression and cell growth. Two different passages of cells were treated with the indicated concentrations of polyamines and each biological sample was measured in triplicates. Error bars represent SEM.

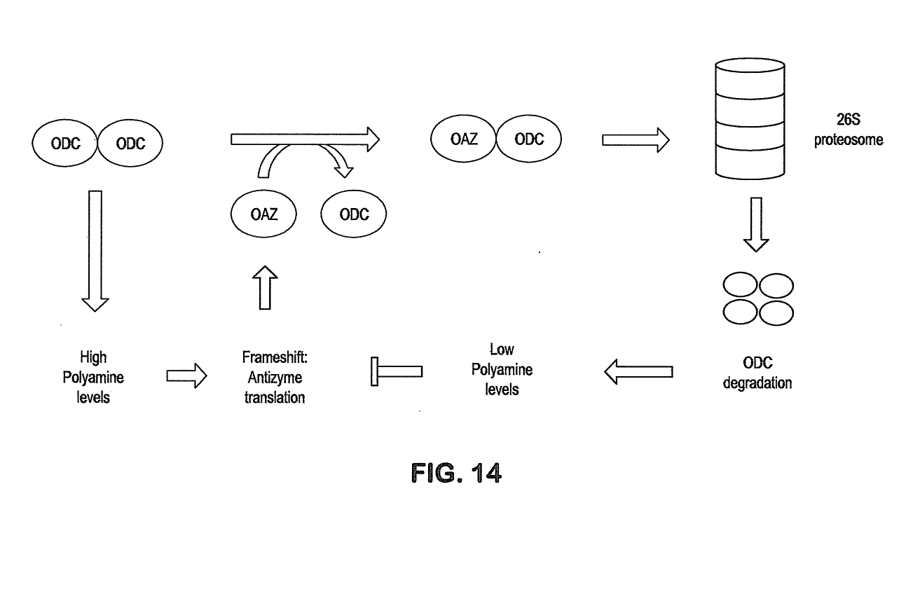

FIG. 14: Schematic diagram of polyamine pathway and regulation of omithine decarboxylase (ODC) by antizyme (OAZ) and antizyme inhibitor (AZIN). Simplified pathway of polyamine synthesis from omithine is indicated by solid arrows and polyamine catabolism by broken arrows. ODC is regulated by OAZ whose translation is turned on by +1 ribosomal frameshifting at a high concentration of polyamines. OAZ is in turn regulated by AZIN, which is an ODC-like protein, but devoid of the enzyme activity.

Results

1. Identification of Genes Whose Silencing Leads to Enhanced Luciferase Expression.

A human genome-wide siRNA screen was conducted in HEK-CMV-Luc2-Hygro cells by using siRNA library targeting 21,585 human genes, with 3 independent arrayed siRNAs per gene. The transfection was done in duplicate: one set of plates was used for measuring the overall luciferase yield and the second set was used for the determination of viable cell density, from which the per cell luciferase yield was calculated (FIG. 8A). The distribution of siRNA activity based on the overall luciferase yield is illustrated in the histogram shown in FIG. 8B, where the red and blue color circle indicates up and down regulation of luciferase expression, respectively. Out of the 64,755 siRNA's tested 1,681 significantly enhanced luciferase expression (MAD-based z-score>3, or 40% to 178% higher than negative control). From the 1,681 siRNAs, 56 genes with at least 2 siRNAs scoring >3 MAD (Table 3) were selected and subjected to follow up evaluation with additional siRNAs. 11,207 or 17.3% of the siRNAs tested that improved per cell luciferase expression by more than 20% (FIG. 8C quadrant I&II), were identified, while only 254 (0.4%) siRNAs achieved more than 20% enhancement in viable cell density (FIG. 8C quadrant I&IV). In this plot, 168 siRNAs associated with the 56 selected genes are indicated by red or yellow circles. Red indicates siRNAs with >3 MAD score, whereas yellow indicates siRNAs with <3 MAD score.

TABLE 3
56 Gene List

| | | | | | SEQ | |
|---|---|---|---|---|---|---|
| | Gene | | | | ID | No. of |
| Symbol | ID | Sequence | VIA | per_cell | NO: | siRNAs |
| 4-Sep | 5414 | GCAGUGGACAUAGAAGAGAtt | 110.8152628 | 140.6578904 | 38 | 2 |
| ABCB8 | 11194 | CGCUUUAACUGGAAGCUCUtt | 103.6615657 | 146.9925887 | 39 | 2 |
| ACSF2 | 80221 | CGAUGUUCGUGGACAUUCUtt | 80.01263817 | 234.6075916 | 40 | 2 |
| ALDH3A2 | 224 | CAACAGUACUUACCGAUGUtt | 115.5131543 | 141.7603494 | 41 | 2 |
| APOBEC3H | 164668 | CAAGUCACCUGUUACCUCAtt | 88.95545951 | 171.8049178 | 42 | 2 |
| C22orf26 | 55267 | GCUAAGUCUUUUCCACAGUtt | 94.5370195 | 155.3854204 | 43 | 2 |
| C3orf19 | 51244 | CAACAGAUCAGAGAACAAAtt | 92.42905679 | 156.4181826 | 44 | 2 |
| CASP8AP2 | 9994 | GGAUAUUGGAGGCUAGUCAtt | 89.67701835 | 157.8682559 | 45 | 3 |
| CCT2 | 10576 | CUCUUAUGGUAACCAAUGAtt | 91.55794321 | 160.2464825 | 46 | 3 |
| CCT7 | 10574 | GUACCUGCGGGAUUACUCAtt | 90.9873026 | 160.6868973 | 47 | 2 |
| CDCA7 | 83879 | GCAAUGCUUGCAAAACUCAtt | 90.97593661 | 189.2523603 | 48 | 2 |
| CHAF1A | 10036 | CGAAACUUGUCAACGGGAAtt | 104.5526978 | 179.2068082 | 49 | 3 |
| CNOT1 | 23019 | GGAGGAAUCUCGAAUGCGAtt | 96.00155804 | 173.2843761 | 50 | 2 |
| DENND5B | 160518 | CCAGCGAUACAACUCCUAUtt | 83.63587838 | 176.8211364 | 51 | 2 |
| EEF1B2 | 1933 | GGAAGAACGUCUUGCACAAtt | 85.47101835 | 183.0712452 | 52 | 2 |
| EEFSEC | 60678 | GAACAAAAUAGACCUCUUAtt | 101.8974711 | 162.6767089 | 53 | 2 |
| FAM102A | 399665 | GCAUCUGUCCGAUCGCUCUtt | 105.5137032 | 143.888826 | 54 | 2 |
| FRZB | 2487 | CAUCAAGCCCUGUAAGUCUtt | 89.58325271 | 163.4311281 | 55 | 2 |
| HNRNPC | 3183 | CAACGGGACUAUUAUGAUAtt | 97.39178755 | 163.4960365 | 56 | 3 |
| HNRPDL | 9987 | GAACGAGUACAGCAAUAUAtt | 108.2138186 | 140.8682863 | 57 | 2 |
| ICA1L | 130026 | ACAGGUCUUUAUCAAAGCAtt | 100.200758515 | 3.4256367 | 58 | 2 |
| INTS1 | 26173 | AGAUCUUUGUCAAGGUGUAtt | 96.25592882 | 153.438416 | 59 | 2 |
| INTS2 | 57508 | GGCGAAUGCUCCUGACUAAtt | 103.8878892 | 156.9770573 | 60 | 2 |
| KAT5 | 10524 | GGACGGAAGCGAAAAUCGAtt | 71.47260615 | 216.2090578 | 61 | 2 |
| KCNJ10 | 3766 | AGGUCAAUGUGACUUUCCAtt | 105.0292112 | 161.7854643 | 62 | 2 |
| KCTD15 | 79047 | CCUGGACAGUUUGAAGCAAtt | 88.00872521 | 174.9462845 | 63 | 2 |
| L3MBTL4 | 91133 | GAUCGUUUGAGAGAACAAAtt | 77.10653003 | 210.3361213 | 64 | 2 |
| MARK2 | 2011 | GCCUAGGAGUUAUCCUCUAtt | 112.2478468 | 137.5542741 | 65 | 2 |
| MFRP | 83552 | GCAACAGAAUCGAGCAAGAtt | 105.5828383 | 154.2862361 | 66 | 2 |
| MGRN1 | 23295 | GGAUGACGAGCUGAACUUUtt | 113.4369441 | 140.1033878 | 67 | 2 |
| MZF1 | 7593 | CAGGUAGUGUAAGCCCUCAtt | 66.87242016 | 302.993981 | 68 | 2 |
| NKX3-2 | 579 | CCCUCCUACUAUUACCCGUtt | 96.86942437 | 162.9994025 | 69 | 2 |
| OAZ1 | 4946 | GAUUAUCCUUGUACUUUGAtt | 101.9042106 | 141.8423185 | 70 | 2 |
| OCRL | 4952 | GAUUACUUCUUGACUAUCAtt | 123.1990206 | 150.8433744 | 71 | 2 |
| OR10P1 | 121130 | GCUCCUCUGUUACCACAGAtt | 91.52564992 | 179.2675123 | 72 | 2 |
| POU5F1 | 5460 | GUCCGAGUGUGGUUCUGUAtt | 85.81980928 | 180.4012462 | 73 | 2 |
| PPP2R1A | 5518 | CUUCGACAGUACUUCCGGAtt | 104.0196693 | 161.6127058 | 74 | 3 |
| PRPF19 | 27339 | GCUCAUCGACAUCAAAGUUtt | 97.00716654 | 170.0884202 | 75 | 2 |
| PRR15 | 222171 | CUUUUAAUGUUAAACUACAtt | 110.9857701 | 133.6010783 | 76 | 2 |
| RAB31 | 11031 | CAAUGGAACAAUCAAAGUUtt | 89.60749662 | 180.7639474 | 77 | 2 |
| RBM22 | 55696 | CGGAAUCAAUGAUCCUGUAtt | 71.39481156 | 212.6523694 | 78 | 2 |
| RDBP | 7936 | AAGUCAACAUAGCCCGAAAtt | 111.4867458 | 145.7069086 | 79 | 2 |
| SART1 | 9092 | CAAUGAUUCUUACCCUCAAtt | 77.16940207 | 257.9289946 | 80 | 2 |
| SF3B3 | 23450 | GUUUCAUCUGGGUUCGCUAtt | 62.65915575 | 233.6485039 | 81 | 2 |
| SF3B4 | 10262 | GUCCUAUCACCGUAUCUUAtt | 55.96204726 | 266.6274525 | 82 | 2 |
| SLC12A8 | 84561 | GCGGAAAAGGUAUCCCUCAtt | 80.17325945 | 184.6083818 | 83 | 2 |
| SNRPB | 6628 | GGCUGUACAUAGUCCUUUUtt | 66.39424587 | 238.4632312 | 84 | 3 |
| SNRPD2 | 6633 | CUGCCGCAACAAUAAGAAAtt | 82.95036211 | 171.1980179 | 85 | 2 |
| SNRPE | 6635 | CAUUGGUUUUGAUGAGUAUtt | 64.38473908 | 226.6587812 | 86 | 2 |
| SNRPF | 6636 | GGUGUAAUAAUGUCCUUUAtt | 79.38554252 | 188.0939999 | 87 | 2 |
| TACC2 | 10579 | GAGCAGAGAUCAUAACCAAtt | 108.2112815 | 141.1042242 | 88 | 2 |
| TBX1 | 6899 | GGAUCACGCAGCUCAAGAUtt | 91.53808265 | 161.6343253 | 89 | 2 |
| U2AF1 | 7307 | GGUGCUCUCGGUUGCACAAtt | 82.3331734 | 187.3622727 | 90 | 3 |
| U2AF2 | 11338 | CAGCAAACCUUUGACCAGAtt | 55.40487046 | 277.5112701 | 91 | 2 |
| ZBTB41 | 360023 | CCAGUUCGACCUGAACAAAtt | 85.75048225 | 176.0187889 | 92 | 2 |
| ZNF358 | 140467 | CAGCCUCACCAAGCACAAAtt | 85.86583978 | 167.4126798 | 93 | 2 |

2. Identification of Pathways Affecting Viable Cell Density and Recombinant Protein Productivity

To identify pathways that affect the reporter protein production, functional ontology analyses were carried out using the 119 siRNAs (Table 4) against the 56 genes (Table 3) that significantly improved the specific luciferase yield, using the PANTHER™ (available on the World Wide Web at pantherdb.org) and AmiGO 2 GOT^{M} browser. The heatmap (FIG. 9) shows that all the siRNAs enhanced per cell luciferase yield (pink to red spectrum), but the majority negatively affected the cell viability (blue shades) which is undesirable in recombinant protein production. The enhancer siRNAs were found to be enriched in the following specific pathways: mRNA processing/spliceosome, transcription, metabolic process, cation transport and protein folding.

TABLE 4
119 siRNA of the 56 genes.

| | | Viable | | | | |
|---|---|---|---|---|---|---|
| SEQ. | | cell | per cell | | | |
| ID. | | density | luciferase | | | |
| NO: | Symbol | (%) | yield (%) | Gene_ID | siRNA sequence | Biological process |
| 94 | APOBEC3H | 66.938 | 227.583 | 164668 | AGAGGCUACUUUGAAAACAtt | RNA processing/ |
| | | | | | | Spliceosome |
| 95 | APOBEC3H | 88.955 | 171.805 | 164668 | CAAGUCACCUGUUACCUCAtt | RNA processing/ |
| | | | | | | Spliceosome |
| 96 | HNRNPC | 73.809 | 227.741 | 3183 | ACACUCUUGUGGUCAAGAAtt | RNA processing/ |
| | | | | | | Spliceosome |
| 97 | HNRNPC | 96.599 | 184.201 | 3183 | GAUGAAGAAUGAUAAGUCAtt | RNA processing/ |
| | | | | | | Spliceosome |
| 98 | HNRNPC | 97.392 | 163.496 | 3183 | CAACGGGACUAUUAUGAUAtt | RNA processing/ |
| | | | | | | Spliceosome |
| 99 | HNRPDL | 75.879 | 227.884 | 9987 | CCCGGAUACUUCUGAAGAAtt | RNA processing/ |
| | | | | | | Spliceosome |
| 100 | HNRPDL | 108.214 | 140.868 | 9987 | GAACGAGUACAGCAAUAUAtt | RNA processing/ |
| | | | | | | Spliceosome |
| 101 | INTS1 | 83.129 | 238.606 | 26173 | GUUCAUCCAUAAGUACAUUtt | RNA processing/ |
| | | | | | | Spliceosome |
| 102 | INTS1 | 96.256 | 153.438 | 26173 | AGAUCUUUGUCAAGGUGUAtt | RNA processing/ |
| | | | | | | Spliceosome |
| 103 | INTS2 | 88.908 | 185.522 | 57508 | GACAUUGGAUCAUACUAAAtt | RNA processing/ |
| | | | | | | Spliceosome |
| 104 | INTS2 | 103.888 | 156.977 | 57508 | GGCGAAUGCUCCUGACUAAtt | RNA processing/ |
| | | | | | | Spliceosome |
| 105 | PRPF19 | 94.579 | 170.349 | 27339 | GCGCAAGCUUAAGAACUUUtt | RNA processing/ |
| | | | | | | Spliceosome |
| 106 | PRPF19 | 97.007 | 170.088 | 27339 | GCUCAUCGACAUCAAAGUUtt | RNA processing/ |
| | | | | | | Spliceosome |
| 107 | RBM22 | 63.879 | 256.443 | 55696 | CCAUAUAUCCGAAUGACCAtt | RNA processing/ |
| | | | | | | Spliceosome |
| 108 | RBM22 | 71.395 | 212.652 | 55696 | CGGAAUCAAUGAUCCUGUAtt | RNA processing/ |
| | | | | | | Spliceosome |
| 109 | SART1 | 51.696 | 275.068 | 9092 | GCAUCGAGGAGACUAACAAtt | RNA processing/ |
| | | | | | | Spliceosome |
| 110 | SART1 | 77.169 | 257.929 | 9092 | CAAUGAUUCUUACCCUCAAtt | RNA processing/ |
| | | | | | | Spliceosome |
| 111 | SF3B3 | 49.133 | 300.611 | 23450 | CAACCUUAUUAUCAUUGAAtt | RNA processing/ |
| | | | | | | Spliceosome |
| 112 | SF3B3 | 62.659 | 233.649 | 23450 | GUUUCAUCUGGGUUCGCUAtt | RNA processing/ |
| | | | | | | Spliceosome |
| 113 | SF3B4 | 35.915 | 409.361 | 10262 | GCAUCAGCUCACAACAAAAtt | RNA processing/ |
| | | | | | | Spliceosome |
| 114 | SF3B4 | 55.962 | 266.627 | 10262 | GUCCUAUCACCGUAUCUUAtt | RNA processing/ |
| | | | | | | Spliceosome |
| 115 | SNRPB | 54.559 | 388.072 | 6628 | AGAUACUGGUAUUGCUCGAtt | RNA processing/ |
| | | | | | | Spliceosome |
| 116 | SNRPB | 53.645 | 370.824 | 6628 | UGGUCUCAAUGACAGUAGAtt | RNA processing/ |
| | | | | | | Spliceosome |
| 117 | SNRPB | 66.394 | 238.463 | 6628 | GGCUGUACAUAGUCCUUUUtt | RNA processing/ |
| | | | | | | Spliceosome |
| 118 | SNRPD2 | 58.901 | 247.842 | 6633 | UGUGGACUGAGGUACCCAAtt | RNA processing/ |
| | | | | | | Spliceosome |
| 119 | SNRPD2 | 82.950 | 171.198 | 6633 | CUGCCGCAACAAUAAGAAAtt | RNA processing/ |
| | | | | | | Spliceosome |
| 120 | SNRPE | 54.945 | 311.516 | 6635 | GGAUCAUGCUAAAAGGAGAtt | RNA processing/ |
| | | | | | | Spliceosome |
| 121 | SNRPE | 64.385 | 226.659 | 6635 | CAUUGGUUUUGAUGAGUAUtt | RNA processing/ |
| | | | | | | Spliceosome |
| 122 | SNRPF | 54.854 | 283.281 | 6636 | AGGGCUAUCUGGUAUCUGUtt | RNA processing/ |
| | | | | | | Spliceosome |
| 123 | SNRPF | 79.386 | 188.094 | 6636 | GGUGUAAUAAUGUCCUUUAtt | RNA processing/ |
| | | | | | | Spliceosome |
| 124 | U2AF1 | 65.083 | 292.710 | 7307 | GAAUAACCGUUGGUUUAAUtt | RNA processing/ |
| | | | | | | Spliceosome |
| 125 | U2AF1 | 63.672 | 240.287 | 7307 | GGAACACUAUGAUGAGUUUtt | RNA processing/ |
| | | | | | | Spliceosome |
| 126 | U2AF1 | 82.333 | 187.362 | 7307 | GGUGCUCUCGGUUGCACAAtt | RNA processing/ |
| | | | | | | Spliceosome |
| 127 | U2AF2 | 54.133 | 311.348 | 11338 | CCAACUACCUGAACGAUGAtt | RNA processing/ |
| | | | | | | Spliceosome |
| 128 | U2AF2 | 55.405 | 277.511 | 11338 | CAGCAAACCUUUGACCAGAtt | RNA processing/ |
| | | | | | | Spliceosome |
| 129 | CNOT1 | 81.492 | 244.722 | 23019 | GCUAUUUCCAGCGAAUAUAtt | Transcription |
| 130 | CNOT1 | 96.002 | 173.284 | 23019 | GGAGGAAUCUCGAAUGCGAtt | Transcription |
| 131 | KAT5 | 70.586 | 284.565 | 10524 | GGAGAAAGAAUCAACGGAAtt | Transcription |
| 132 | KAT5 | 71.473 | 216.209 | 10524 | GGACGGAAGCGAAAAUCGAtt | Transcription |
| 133 | L3MBTL4 | 63.577 | 250.367 | 91133 | GAACUUCAAUGGAAAACAUtt | Transcription |
| 134 | L3MBTL4 | 77.107 | 210.336 | 91133 | GAUCGUUUGAGAGAACAAAtt | Transcription |
| 135 | MZF1 | 66.872 | 302.994 | 7593 | CAGGUAGUGUAAGCCCUCAtt | Transcription |
| 136 | MZF1 | 49.189 | 299.886 | 7593 | AGGUUACAGAGGACUCAGAtt | Transcription |
| 137 | NKX3-2 | 88.616 | 212.905 | 579 | GAACCGUCGCUACAAGACAtt | Transcription |
| 138 | NKX3-2 | 96.869 | 162.999 | 579 | CCCUCCUACUAUUACCCGUtt | Transcription |
| 139 | POU5F1 | 83.011 | 194.043 | 5460 | GGAGAUAUGCAAAGCAGAAtt | Transcription |
| 140 | POU5F1 | 85.820 | 180.401 | 5460 | GUCCGAGUGUGGUUCUGUAtt | Transcription |
| 141 | RDBP | 97.298 | 183.386 | 7936 | AGAGGACCCAGAUUGUCUAtt | Transcription |
| 142 | RDBP | 111.487 | 145.707 | 7936 | AAGUCAACAUAGCCCGAAAtt | Transcription |
| 143 | TBX1 | 75.599 | 193.846 | 6899 | GCAAAGAUAGCGAGAAAUAtt | Transcription |
| 144 | TBX1 | 91.538 | 161.634 | 6899 | GGAUCACGCAGCUCAAGAUtt | Transcription |
| 145 | ZBTB41 | 85.750 | 176.019 | 360023 | CCAGUUCGACCUGAACAAAtt | Transcription |
| 146 | ZBTB41 | 83.351 | 171.308 | 360023 | GACCUAUACUCAUUCUGCAtt | Transcription |
| 147 | ZNF358 | 61.490 | 256.085 | 140467 | GUUUCGACCUCGAUCCAGAtt | Transcription |
| 148 | ZNF358 | 85.866 | 167.413 | 140467 | CAGCCUCACCAAGCACAAAtt | Transcription |
| 149 | ABCB8 | 57.472 | 294.800 | 11194 | CGACCAUCAUGGAAAACAUtt | Metabolic process |
| 150 | ABCB8 | 103.662 | 146.993 | 11194 | CGCUUUAACUGGAAGCUCUtt | Metabolic process |
| 151 | ACSF2 | 59.084 | 288.857 | 80221 | GAAACUGCAUGAGAAGACAtt | Metabolic process |
| 152 | ACSF2 | 80.013 | 234.608 | 80221 | CGAUGUUCGUGGACAUUCUtt | Metabolic process |
| 153 | ALDH3A2 | 87.831 | 171.072 | 224 | CACUUUCCUGGGUAUUGUAtt | Metabolic process |
| 154 | ALDH3A2 | 115.513 | 141.760 | 224 | CAACAGUACUUACCGAUGUtt | Metabolic process |
| 155 | OAZ1 | 94.839 | 169.972 | 4946 | GCCUUGCUCCGAACCUUCAtt | Metabolic process |
| 156 | OAZ1 | 101.904 | 141.842 | 4946 | GAUUAUCCUUGUACUUUGAtt | Metabolic process |
| 157 | PPP2R1A | 60.645 | 254.007 | 5518 | GAACAGCUGGGAACCUUCAtt | Metabolic process |
| 158 | PPP2R1A | 104.020 | 161.613 | 5518 | CUUCGACAGUACUUCCGGAtt | Metabolic process |
| 159 | PPP2R1A | 96.357 | 157.243 | 5518 | GGAGUUCUUUGAUGAGAAAtt | Metabolic process |
| 160 | 4-Sep | 68.989 | 220.902 | 5414 | GGACCAAGCCCUAAAGGAAtt | Metabolic process |
| 161 | 4-Sep | 110.815 | 140.658 | 5414 | GCAGUGGACAUAGAAGAGAtt | Metabolic process |
| 162 | DENND5B | 76.131 | 235.013 | 160518 | CGAUAUGCUUUUCUACGUUtt | Cation transport |
| 163 | DENND5B | 83.636 | 176.821 | 160518 | CCAGCGAUACAACUCCUAUtt | Cation transport |
| 164 | KCNJ10 | 83.512 | 259.478 | 3766 | GCAGGCACAUGGUUCCUCUtt | Cation transport |
| 165 | KCNJ10 | 105.029 | 161.785 | 3766 | AGGUCAAUGUGACUUUCCAtt | Cation transport |
| 166 | KCTD15 | 76.798 | 186.195 | 79047 | CCAAGUCCAAUGCACCUGUtt | Cation transport |
| 167 | KCTD15 | 88.009 | 174.946 | 79047 | CCUGGACAGUUUGAAGCAAtt | Cation transport |
| 168 | SLC12A8 | 78.156 | 185.175 | 84561 | GCUUCCUCUUGGACCUCAAtt | Cation transport |
| 169 | SLC12A8 | 80.173 | 184.608 | 84561 | GCGGAAAAGGUAUCCCUCAtt | Cation transport |
| 170 | CASP8AP | 267.320 | 284.062 | 9994 | CCAACAAGGAAGACGAAAAtt | Apoptotic process |
| 171 | CASP8AP | 279.253 | 272.683 | 9994 | CCCUGUUCAUUAUAAGUCUtt | Apoptotic process |
| 172 | CASP8AP | 289.677 | 157.868 | 9994 | GGAUAUUGGAGGCUAGUCAtt | Apoptotic process |
| 173 | CDCA7 | 82.689 | 209.398 | 83879 | GACUAUUGAUACCAAAACAtt | Apoptotic process |
| 174 | CDCA7 | 90.976 | 189.252 | 83879 | GCAAUGCUUGCAAAACUCAtt | Apoptotic process |
| 175 | CCT2 | 85.046 | 189.850 | 10576 | CAUUGGUGUUGACAAUCCAtt | Protein folding |
| 176 | CCT2 | 79.093 | 186.786 | 10576 | GUUGCAAACUUAUCGAGGAtt | Protein folding |
| 177 | CCT2 | 91.558 | 160.246 | 10576 | CUCUUAUGGUAACCAAUGAtt | Protein folding |
| 178 | CCT7 | 76.280 | 200.322 | 10574 | AAAUGCAACCCAAAAAGUAtt | Protein folding |
| 179 | CCT7 | 90.987 | 160.687 | 10574 | GUACCUGCGGGAUUACUCAtt | Protein folding |
| 180 | C22orf26 | 93.181 | 185.763 | 55267 | CCACCCUACUAUGUACUGUtt | Miscellaneous |
| 181 | C22orf26 | 94.537 | 155.385 | 55267 | GCUAAGUCUUUUCCACAGUtt | Miscellaneous |
| 182 | C3orf19 | 91.714 | 202.960 | 51244 | CAGUUACUUUCAAAACUCUtt | Miscellaneous |
| 183 | C3orf19 | 92.429 | 156.418 | 51244 | CAACAGAUCAGAGAACAAAtt | Miscellaneous |
| 184 | CHAF1A | 82.390 | 228.483 | 10036 | GCCUGAAUCUUGUCCCAAAtt | Miscellaneous |
| 185 | CHAF1A | 66.511 | 215.853 | 10036 | GAAGAAGACUCUGUACUCAtt | Miscellaneous |
| 186 | CHAF1A | 104.553 | 179.207 | 10036 | CGAAACUUGUCAACGGGAAtt | Miscellaneous |
| 187 | EEF1B2 | 75.570 | 200.070 | 1933 | AGAAAGCUUUGGGCAAAUAtt | Miscellaneous |
| 188 | EEF1B2 | 85.471 | 183.071 | 1933 | GGAAGAACGUCUUGCACAAtt | Miscellaneous |
| 189 | EEFSEC | 101.897 | 162.677 | 60678 | GAACAAAAUAGACCUCUUAtt | Miscellaneous |
| 190 | EEFSEC | 95.368 | 152.850 | 60678 | CUGUGGAAAAGAUACCGUAtt | Miscellaneous |
| 191 | FAM102A | 66.121 | 214.156 | 399665 | GCCCACUAUUCUCAGCUCAtt | Miscellaneous |
| 192 | FAM102A | 105.514 | 143.889 | 399665 | GCAUCUGUCCGAUCGCUCUtt | Miscellaneous |
| 193 | FRZB | 74.811 | 205.132 | 2487 | GGGACACUGUCAACCUCUAtt | Miscellaneous |
| 194 | FRZB | 89.583 | 163.431 | 2487 | CAUCAAGCCCUGUAAGUCUtt | Miscellaneous |
| 195 | ICA1L | 67.947 | 258.104 | 130026 | UGAAGAUAAUCGAGAAAUAtt | Miscellaneous |
| 196 | ICA1L | 100.201 | 153.426 | 130026 | ACAGGUCUUUAUCAAAGCAtt | Miscellaneous |
| 197 | MARK2 | 95.471 | 162.372 | 2011 | GACUCAGAGUAACAACGCAtt | Miscellaneous |
| 198 | MARK2 | 112.248 | 137.554 | 2011 | GCCUAGGAGUUAUCCUCUAtt | Miscellaneous |
| 199 | MFRP | 88.359 | 164.639 | 83552 | CUAACUACCCAGACCCUUAtt | Miscellaneous |
| 200 | MFRP | 105.583 | 154.286 | 83552 | GCAACAGAAUCGAGCAAGAtt | Miscellaneous |
| 201 | MGRN1 | 63.690 | 273.535 | 23295 | CCCUGAAGGUUACCUCUUUtt | Miscellaneous |
| 202 | MGRN1 | 113.437 | 140.103 | 23295 | GGAUGACGAGCUGAACUUUtt | Miscellaneous |
| 203 | OCRL | 123.199 | 150.843 | 4952 | GAUUACUUCUUGACUAUCAtt | Miscellaneous |
| 204 | OCRL | 109.377 | 136.174 | 4952 | CUCCCGCAGUUGAACAUCAtt | Miscellaneous |
| 205 | OR10P1 | 61.943 | 281.113 | 121130 | CUCUGAUUGUCACCUCUUAtt | Miscellaneous |
| 206 | OR10P1 | 91.526 | 179.268 | 121130 | GCUCCUCUGUUACCACAGAtt | Miscellaneous |
| 207 | PRR15 | 102.629 | 146.756 | 222171 | CGCUCACCAACAGCAGAAAtt | Miscellaneous |
| 208 | PRR15 | 110.986 | 133.601 | 222171 | CUUUUAAUGUUAAACUACAtt | Miscellaneous |
| 209 | RAB31 | 84.418 | 228.634 | 11031 | GAACUUCACAAGUUCCUCAtt | Miscellaneous |
| 210 | RAB31 | 89.607 | 180.764 | 11031 | CAAUGGAACAAUCAAAGUUtt | Miscellaneous |
| 211 | TACC2 | 82.634 | 188.258 | 10579 | GGAUUACAGAAACUCCUAUtt | Miscellaneous |
| 212 | TACC2 | 108.211 | 141.104 | 10579 | GAGCAGAGAUCAUAACCAAtt | Miscellaneous |

3. Selection Often Genes Whose Silencing Leads to Enhanced Luciferase Expression

For selecting gene candidates for further work, three additional siRNAs were tested for each of the 56 target genes identified from the primary screen. From the combined data of the primary and the validation screen of the 56 genes, ten genes were selected, based on the criteria that least 3 out of 6 siRNAs tested displayed a MAD-based z-scores higher than 3.0 (Table 5). The viable cell number was also taken into consideration to remove candidates with significant toxicity. The median value of the overall luciferase yield for each selected gene calculated from the 6 siRNAs was improved by 24% to 72% compared with negative control, and the median of MAD-based z-scores ranged from 2.13 to 4.55.

TABLE 5
Confirmed top 10 genes with 3 or more siRNAs yielding >50% increase in luciferase activity. A 50% increase
is biologically relevant and also corresponds to high statistical significance (>3 MAD-based z-scores).

| | | Overall | MAD- | |
|---|---|---|---|---|
| | | luciferase | based | |
| Gene | Description | yield (%)*^{,†} | z-score* | Function |
| INTS1 | Integrator Complex | 172 | 4.55 | 3′- end processing of small nuclear RNAs |
| | Subunit 1 | | | U1 and U2 |
| INTS2 | Integrator Complex | 165 | 4.17 | 3′- end processing of small nuclear RNAs |
| | Subunit 2 | | | U1 and U2 |
| HNRNPC | Heterogeneous Nuclear | 163 | 4.10 | Influencing pre-mRNA processing and other |
| | Ribonucleoprotein | | | aspects of mRNA metabolism and transport |
| CASP8AP2 | Caspase 8 Associated | 156 | 3.70 | Activation and regulation of CASP8 in FAS- |
| | Protein 2 | | | mediated apoptosis |
| OAZ1 | Ornithine Decarboxylase | 153 | 3.57 | Inhibiting ornithine decarboxylase and |
| | Antizyme | | | inactivating the polyamine uptake transporter |
| PPP2R1A | Protein Phosphatase 2, | 153 | 3.56 | Serving as a scaffold for Protein Phosphatase |
| | Regulatory Subunit A, | | | 2 assembly, essential for signal transduction |
| | Alpha | | | pathways |
| PRPF19 | Pre-mRNA Processing | 147 | 3.27 | Spliceosome assembly and activating pre- |
| | Factor 19 | | | mRNA splicing |
| CHAF1A | Chromatin Assembly | 138 | 2.80 | mediating chromatin assembly in DNA |
| | Factor 1, Subunit A | | | replication and DNA repair |
| CCT2 | Chaperonin Containing | 126 | 2.23 | Chaperonin-mediated protein folding of |
| | TCP1, Subunit 2 (Beta) | | | actin, tubulin and other proteins |
| EEF1B2 | Eukaryotic Translation | 124 | 2.13 | exchanging GDP bound to EF-1-α to GTP |
| | Elongation Factor 1 | | | during the transfer of aminoacylated tRNAs |
| | Beta 2 | | | to the ribosome |

*All values are medians of result from 6 siRNAs(3 siRNAs in primary screen and 3 siRNAs in validation screen) targeting a top gene.
^{†}Values are normalized to negative control siN.C. transfected cells (set as 100%) .

Four out of the ten target genes, INTS1, INTS2, HNRNPC, and PRPF19, are involved in mRNA splicing process; they encode important proteins for spliceosome formation, such as integrator complex, heterogeneous nuclear ribonucleoprotein and pre-mRNA processing factor 19. The remainder of the identified genes encodes proteins involved in a wide span of biological functions, including cell growth and division, signal transduction, apoptosis, regulation of cellular polyamine concentration and protein translation and folding.

4. Effects of Silencing the Ten Target Genes on Secreted and Membrane Protein Production

To examine the silencing effect of the 10 selected genes on the expression of other recombinant proteins from HEK293 cells, three additional cell lines were tested: 1) HEK-GPC3-hFc cell line, which constitutively secretes glypican −3 hFc-fusion protein (GPC3-hFc) as a representative of antibody secreting cell lines, 2) T-REx-293-NTSR1-GFP cell line constructed previously for the production of functional neurotensin receptor type I (NTSR1), and 3) T-REx-293-SERT-GFP cell line, an inducible cell line for high level expression of serotonin transporter (SERT), a hard-to-express 12 transmembrane domain protein. Both NTRS1 and SERT were fused with GFP at the C-terminus, allowing proximal protein quantification by flow cytometry. As shown in FIG. 10, the siRNAs against the ten selected genes exhibited varying effects on the expression of the secreted and the membrane proteins. The silencing of INTS1, HNRHPC, OAZ1 and PPP2R1A consistently improved the expression of all reporter proteins tested. However, the silencing of INTS1 and HNRNPC led to a significantly reduced viable cell number, an indication that these genes may be essential for cell survival or cell growth. Silencing of the OAZ1 and PPP2R1A genes showed minimal negative effects on the viable cell number.

5. Effect of Silencing OAZ1 on Luciferase Expression.

Among the selected genes, the antizyme 1 (OAZ1) was chosen for follow-up studies since its silencing consistently improved cytosolic, secreted and membrane protein expression and caused minimal growth disadvantage in the four cell lines tested (FIG. 10A). Five of the six OAZ1 siRNAs tested (Table 6) enhanced luciferase production (luciferase activity (%)) by 28-74%, and OAZ1 siRNA5 was chosen for the rest of the study. Unlike OAZ1 siRNAs, the siRNAs against antizyme isoforms OAZ2 (a minor isoform) and OAZ3 (a testis specific form) caused no significant enhancement of luciferase production.

TABLE 6
The list of siRNAs targeting the polyamine
pathway genes, OAZ1, OAZ2, OAZ3, ODC and AZIN1
and their effects on luciferase activity,
cell viability and per cell luciferase yield.
The data are from the primary siRNA screen,
except for the last three additional siRNAs
against OAZ1.

| | | | | | Per cell |
|---|---|---|---|---|---|
| Gene | | SEQ ID | Luciferase | Viable cell | luciferase |
| Symbol | siRNA sequence | NO: | activity (%) | number (%) | yield (%) |
| OAZ1 | GCCUUGCUCCGAACCUUCAtt | 155 | 161.1 | 94.8 | 169.9 |
| | GAUUAUCCUUGUACUUUGAtt | 156 | 144.5 | 101.9 | 141.8 |
| | GGCUGAAUGUAACAGAGGAtt | 213 | 127.6 | 94.9 | 134.5 |
| | CCGUAGACUCGCUCAUCUCtt | 214 | 174.4 | 85.4 | 204.2 |
| | GCUAACUUAUUCUACUCCGtt | 215 | 171.1 | 110.6 | 154.7 |
| | GGGAAUAGUCAGAGGGAUCtt | 216 | 92.8 | 102.7 | 90.4 |
| OAZ2 | ACAUCGUCCACUUCCAGUAtt | 217 | 97.4 | 96.3 | 101.1 |
| | GGACCUCCCUGUGAAUGAUtt | 218 | 95.4 | 86.0 | 110.9 |
| | CAGAUGGAUUAUUAGCUGAtt | 219 | 94.9 | 105.4 | 90.0 |
| OAZ3 | CCGGGAAAGUUUGACUGCAtt | 220 | 101.5 | 75.8 | 133.9 |
| | CCACGACCAGCUUAAAGAAtt | 221 | 90.5 | 95.76 | 94.5 |
| | GACUUUCACUUCCGCCUUAtt | 222 | 74.3 | 87.7 | 84.7 |
| ODC1 | GAUGACUUUUGAUAGUGAAtt | 223 | 18.0 | 56.1 | 32.1 |
| | GCAUGUAUCUGCUUGAUAUtt | 224 | 20.0 | 50.7 | 39.4 |
| | GCUUGCAGUUAAUAUCAUUtt | 225 | 28.4 | 60.8 | 46.7 |
| AZIN1 | CACUCGCAGUUAAUAUCAUtt | 226 | 25.2 | 64.6 | 39.0 |
| | CGAUGAACAUGUUAGACAUtt | 227 | 30.4 | 72.1 | 42.2 |
| | GCCCUCUGUUGGAUAUCUAtt | 228 | 45.6 | 72.1 | 63.2 |

As cells transfected with siOAZ1 showed significantly higher luciferase production for an extended period of time (FIG. 11A), the efficacies of silencing antizyme 1 was evaluated with qRT-PCR (FIG. 11B). The expression of OAZ1 mRNA in the 24-72 hour period following the transfection of siRNA, was less than 3% compared with negative control siRNA-transfected cells, confirming the silencing by the siRNA. Throughout the 96 hour period luciferase mRNA levels did not increase and remained somewhat lower than those of negative control cells (FIG. 11C), an indication that the enhanced luciferase production is the result of an increased translation.

6. Effect of Silencing OAZ1 on Ornithine Decarboxylase and Cellular Polyamines

OAZ1 is a negative regulator of the ODC, a rate-limiting enzyme in the polyamine biosynthesis (FIG. 14). OAZ1 inactivates ODC by forming heterodimers with the ODC monomer and by directing the protein to degradation by the 26S proteasome. OAZ1 itself is regulated by antizyme inhibitor (AZIN), an ODC-like protein that increase the ODC concentration as a result of reducing OAZ (FIG. 14). As seen in FIG. 12A silencing OAZ1 with siRNA increased significantly the ODC level from 24 to 96 hours, whereas little or no change in ODC was observed in the un-transfected and siN.C-transfected cells. The elevated ODC is apparently not the result of enhanced ODC transcription, since qRT-PCR analysis demonstrated consistent reduction of ODC mRNA levels after silencing the OAZ1 (FIG. 12B). As seen in FIG. 5C silencing OAZ1 caused changes in cellular polyamine levels; the putrescine concentration was 10 fold higher compared with the negative control cells. Spermidine concentration was increased to a lesser extent, whereas spermine was either unchanged or reduced.

7. Effects of Exogenous Polyamines on Luciferase Protein Expression

Increased cellular polyamines in OAZ1-silenced cells are most likely responsible for the enhanced cellular production of the reporter proteins. To further verify this, the impacts of exogenously added polyamines on luciferase expression level and viable cell number were determined. As can be seen in FIG. 13A, up to 40% increase of luciferase expression was observed when putrescine was added to medium at 100 μM and 10% enhanced growth was observed with putrescine addition at 50 μM. Higher concentrations did not lead to further enhancement of luciferase production. The spermidine effect is seen in FIG. 13B; 36% increase in luciferase expression was observed at 20 μM, and 24% increase in cell growth was achieved at 10 μM. In case of spermine addition, only 16% increase in luciferase expression was observed at 10 μM and higher concentrations caused reduction in both luciferase expression and viable cell (FIG. 13C). The inhibitory effects of spermidine (>100 μM) and spermine (>20 μM) are probably due to generation of the-toxic oxidation products by ruminant serum oxidases present in the culture medium.

Discussion

Cultivated mammalian cells are the dominant vehicle for production of recombinant proteins for bio-therapeutics and structural studies. As a result, continuous effort has been directed toward improving cellular production capabilities. Previous work demonstrated the ability to improve recombinant protein expression based primarily on previous knowledge of specific genes and pathways, but there is a need for discovering novel genes and pathways for improved production. In order to discover new candidates suitable for improving recombinant protein production from HEK 293 cells, an extensive, high throughput RNA interference (RNAi) screen was performed. Genome-wide RNAi screening has emerged as a powerful tool for probing gene functions and for target discovery in various diseases. However, it has rarely been used for identifying targets for enhanced recombinant protein production. The purpose of the present study was to identify genes that showed improved recombinant protein production following their down regulation.

An HEK293 cell line expressing the luciferase reporter was subjected to interference with 64, 755 siRNAs targeting 21,585 human genes. Approximately 2.6% of the library (1,681 siRNAs) strongly improved the luciferase expression with a MAD-based z-score >3. To eliminate the introduction of ‘false positives’ by off-target effects, gene hits were considered ‘true positive’ only if more than two single siRNAs targeting the gene passed the MAD-based z-score >3. Fifty six genes were selected and were subjected to a validation screen with 3 additional siRNAs for each gene. From the data generated by the six siRNAs for each of the 56 genes, ten genes were selected for further analysis. Only those genes that showed an increase in luciferase yield of 3 MAD-based z-scores by 3 or more siRNAs were chosen. This high statistical significance also corresponds to 40% increase in luciferase activity.

The influences of the siRNAs targeting the ten identified genes on recombinant protein expression from the HEK cells were evaluated further by measuring the expression of three additional recombinant proteins: a secreted protein (GPC3-hFc) and two “hard”-to-express membrane proteins (neurotensin receptor type I and serotonin transporter). Silencing of the INTS1, HNRHPC, OAZ1, and PPP2R1A consistently improved production of all the reporter proteins. Of these four genes, silencing INTS1 or HNRHPC also affected cell viability. From the other two genes that slightly affected the cell viability, OAZ1 was chosen for follow-up studies. The identification of OAZ1 as a gene whose silencing can enhance reporter protein production is an indication that this gene normally suppresses protein synthesis. This is compatible with the known function of the antizyme as a negative regulator of polyamine homeostasis, cell proliferation and transformation. The current findings suggest that increased concentration of cellular putrescine and spermidine increases the biosynthesis of the reporter proteins without increasing their transcription, and, therefore, provide new insights into the primary function of polyamines in the regulation of translation. Consistent with this observation is published information that depleting cellular spermidine and spermine by over expressing spermidine/spermine N1-acetyltransferase 1 (SSAT1) led to suppression of protein biosynthesis without inhibiting DNA and RNA biosynthesis.

Although the invention has been described with reference to the examples herein, it will be understood that modifications and variations are encompassed within the spirit and scope of the invention. Accordingly, the invention is limited only by the following claims.

What is claimed is:

1. A method of increasing production of a protein of interest in a cell comprising contacting the cell with an miRNA, siRNA or combination thereof under conditions wherein the miRNA or siRNA is incorporated into the cell, wherein an increase in production of the protein greater than that of a control cell not contacted with the miRNA or siRNA is indicative of increased protein production in the cell, thereby increasing production of the protein of interest in the cell.

2. The method of claim 1, wherein the cell is a mammalian cell.

3. The method of claim 2, wherein the cell is an HEK or CHO cell.

4. The method of claim 1, wherein the cell transiently expresses the miRNA or siRNA.

5. The method of claim 1, wherein the cell stably expresses the miRNA or siRNA.

6. The method of claim 1, wherein the protein is a cytosolic, intracellular, secreted or membrane protein.

7. The method of claim 1, wherein the protein production is increased greater than 1.1, 1.2, 1.3. 1.4, 1.5, 1.6, 1.7, 1.8, 1.9, 2.0 times or more as compared to the control cell not contacted with the miRNA or siRNA.

8. The method of claim 1, wherein the miRNA is one or more miRNAs comprising a sequence selected from the group consisting of SEQ ID NOs: 1-26, and any combination thereof.

9. The method of claim 8, wherein the miRNA is one or more miRNAs comprising a sequence selected from the group consisting of SEQ ID NOs:1-4, 20, 21, 25, and any combination thereof.

10. The method of claim 9, wherein the miRNA is a plurality of miRNAs, each having a sequence as set forth in SEQ ID NOs:2, 3, 20, 21 or 25.

11. The method of claim 1, wherein the miRNA comprises a sequence as set forth in SEQ ID NO:28 or SEQ ID NO:29.

12. The method of claim 11, wherein the miRNA comprises a sequence as set forth in SEQ ID NO:28, and is selected from the group consisting of SEQ ID NOs:4, 16 and 22.

13. The method of claim 1, wherein the siRNA is one or more siRNAs that inhibits expression of a gene set forth in Table 3.

14. The method of claim 13, wherein the siRNA is one or more siRNAs having a sequence selected from the group consisting of SEQ ID NOs:38-212.

15. The method of claim 13, wherein the siRNA is one or more siRNAs that inhibits INTS1, INTS2, HNRNPC, CASP8AP2, OAZ1, ODC1, AZIN1, PPP2R1A, PRPF19, CHAF1A, CCT2, EEF1B2, and any combination thereof.

16. The method of claim 15, wherein the siRNA inhibits OAZ1.

17. The method of claim 16, wherein the siRNA has a sequence set forth in SEQ ID NO:155 or SEQ ID NO:156.

18. The method of claim 1, wherein the cell in contacted with at least one miRNA and at least one siRNA.

19. The method of claim 1, wherein the at least one miRNA has a sequence selected from SEQ ID NOs: 1-26, and the at least one siRNA has a sequence selected from SEQ ID NOs:38-212.

20. The method of claim 1, further comprising harvesting the protein of interest.

21. An isolated nucleic acid sequence comprising a heterologous promoter operably linked to a miRNA sequence, the miRNA sequence having from about 6-25 nucleotides, wherein the miRNA sequence comprises a sequence as set forth in SEQ ID NOs:1-26.

22. A vector comprising the nucleic acid of claim 21.

23. The vector of claim 22, wherein the vector comprises an origin of replication, a selectable marker, a reporter gene, a cloning site, or any combination thereof.

24. An isolated nucleic acid sequence comprising a heterologous promoter operably linked to a miRNA sequence, the miRNA sequence having from about 6-25 nucleotides, wherein the miRNA sequence comprises a sequence as set forth in SEQ ID NO:28 or SEQ ID NO:29.

25. A vector comprising the nucleic acid of claim 24.

26. The vector of claim 25, wherein the vector comprises an origin of replication, a selectable marker, a reporter gene, a cloning site, or any combination thereof.

27. A cell comprising the nucleic acid sequence of claim 21 or claim 24.

28. A method of identifying a miRNA for enhancing expression of a protein comprising:
a) contacting a cell comprising a detectably labeled protein with a plurality of miRNAs; and
b) measuring protein production in a cell contacted with or not contacted with the miRNAs, and comparing the protein production in each cell, wherein an increase in expression of the protein in the cell contacted with the miRNA is indicative of an miRNA which enhances expression of the protein, thereby identifying the miRNA.

29. The method of claim 28, further comprising assessing the functionality of the enhanced protein produced.

30. The method of claim 28, wherein the plurality of miRNAs are transiently transfected to the cell comprising the detectably labeled protein.

31. The method of claim 28, wherein the detectable label comprises luciferase (LUC), β-lactamase, chloramphenicol acetyltransferase (CAT), adenosine deaminase (ADA), aminoglycoside phosphotransferase (neo, G418), dihydrofolate reductase (DHFR), hygromycin-B-phosphotransferase (HPH), thymidine kinase (TK), β-galactosidase (β-gal), and xanthine guanine phosphoribosyltransferase (XGPRT), an affinity or epitope tag, or a fluorescent protein.

32. The method of claim 31, wherein the detectable label is a fluorescent protein.

33. The method of claim 32, wherein the fluorescent protein is green fluorescent protein (GFP) or enhanced green fluorescent protein (eGFP)

34. The method of claim 28, wherein detection of the detectable label is performed using fluorescence microscopy.

35. The method of claim 28, wherein the method is performed in a high throughput format.

36. A kit comprising:
a) a miRNA having a sequence from about 6-25 nucleotides, wherein the miRNA sequence comprises a sequence as set forth in SEQ ID NOs: 1-26; and
b) a siRNA, wherein the siRNA inhibits expression of a gene set forth in Table 3.

37. The kit of claim 36, wherein the siRNAs has a sequence selected from the group consisting of SEQ ID NOs:38-212.

38. A kit comprising a reagent for inhibiting or silencing a gene listed in Table 3 for increasing protein production in a cell.

39. The kit of claim 38, further comprising a miRNA having a sequence from about 6-25 nucleotides, wherein the miRNA sequence comprises a sequence as set forth in SEQ ID NOs:1-26.

40. The kit of claim 38, wherein the reagent is used to accomplish a genome editing methodology comprising a Crispr, zinc finger nuclease, or transcription activator-like effector nuclease (Talen).

41. A method of increasing production of a protein of interest in a cell comprising inhibiting or silencing one or more genes as listed in Table 3, wherein an increase in production of the protein greater than that of a control cell in which the one or more genes is not inhibited or silenced is indicative of increased protein production in the cell.

42. The method of claim 41, wherein the cell is a mammalian cell.

43. The method of claim 42, wherein the cell is an HEK or CHO cell.

44. The method of claim 41, wherein the protein is a cytosolic, intracellular, secreted or membrane protein.

45. The method of claim 41, wherein the protein production is increased greater than 1.1, 1.2, 1.3. 1.4, 1.5, 1.6, 1.7, 1.8, 1.9, 2.0 times or more as compared to the control cell.

45. The method of claim 41, wherein the gene is INTS1, INTS2, HNRNPC, CASP8AP2, OAZ1, ODC1, AZIN1, PPP2R1A, PRPF19, CHAF1A, CCT2, EEF1B2, and any combination thereof.

46. The method of claim 45, wherein the gene is OAZ1.

47. The method of claim 41, wherein silencing or inhibition is achieved via a genome editing methodology.

48. The method of claim 47, wherein the genome editing methodology comprising a Crispr, zinc finger nuclease, or transcription activator-like effector nuclease (Talen).

49. The method of claim 41, wherein expression of the gene is knocked-out or knocked-down.

50. The method of claim 41, wherein silencing or inhibition results from deletion or mutation of the gene.


SEQUENCE LISTING (228 sequences)

SEQ ID NO: 1 - RNA, 22 nt, Artificial Sequence
  Homo sapiens
accuggcauacaauguagauuu

SEQ ID NO: 2 - RNA, 22 nt, Artificial Sequence
  Homo sapiens
uaauacugucugguaaaaccgu

SEQ ID NO: 3 - RNA, 22 nt, Artificial Sequence
  Homo sapiens
aguucuucaguggcaagcuuua

SEQ ID NO: 4 - RNA, 22 nt, Artificial Sequence
  Homo sapiens
cacuggcuccuuucuggguaga

SEQ ID NO: 5 - RNA, 23 nt, Artificial Sequence
  Homo sapiens
ugguuguaguccgugcgagaaua

SEQ ID NO: 6 - RNA, 22 nt, Artificial Sequence
  Homo sapiens
cugugcgugugacagcggcuga

SEQ ID NO: 7 - RNA, 22 nt, Artificial Sequence
  Homo sapiens
cuauacagucuacugucuuucc

SEQ ID NO: 8 - RNA, 21 nt, Artificial Sequence
  Homo sapiens
acucuuucccuguugcacuac

SEQ ID NO: 9 - RNA, 21 nt, Artificial Sequence
  Homo sapiens
caucccuugcaugguggaggg

SEQ ID NO: 10 - RNA, 23 nt, Artificial Sequence
  Homo sapiens
cagugcaauaguauugucaaagc

SEQ ID NO: 11 - RNA, 21 nt, Artificial Sequence
  Homo sapiens
cuuuuugcggucugggcuugc

SEQ ID NO: 12 - RNA, 20 nt, Artificial Sequence
  Homo sapiens
guguguggaaaugcuucugc

SEQ ID NO: 13 - RNA, 22 nt, Artificial Sequence
  Homo sapiens
ugagguaguagguuguaugguu

SEQ ID NO: 14 - RNA, 21 nt, Artificial Sequence
  Homo sapiens
ugagugccggugccugcccug

SEQ ID NO: 15 - RNA, 22 nt, Artificial Sequence
  Homo sapiens
gcuacuucacaacaccagggcc

SEQ ID NO: 16 - RNA, 22 nt, Artificial Sequence
  Homo sapiens
aacuggcccucaaagucccgcu

SEQ ID NO: 17 - RNA, 21 nt, Artificial Sequence
  Homo sapiens
aggaggcagcgcucucaggac

SEQ ID NO: 18 - RNA, 23 nt, Artificial Sequence
  Homo sapiens
aucgcugcgguugcgagcgcugu

SEQ ID NO: 19 - RNA, 22 nt, Artificial Sequence
  Homo sapiens
acagauucgauucuaggggaau

SEQ ID NO: 20 - RNA, 22 nt, Artificial Sequence
  Homo sapiens
uuggggaaacggccgcugagug

SEQ ID NO: 21 - RNA, 22 nt, Artificial Sequence
  Homo sapiens
aagcugccaguugaagaacugu

SEQ ID NO: 22 - RNA, 22 nt, Artificial Sequence
  Homo sapiens
aacuggccuacaaagucccagu

SEQ ID NO: 23 - RNA, 22 nt, Artificial Sequence
  Homo sapiens
uccgucucaguuacuuuauagc

SEQ ID NO: 24 - RNA, 22 nt, Artificial Sequence
  Homo sapiens
aaaccuguguuguucaagaguc

SEQ ID NO: 25 - RNA, 23 nt, Artificial Sequence
  Homo sapiens
uaaggugcaucuagugcagauag

SEQ ID NO: 26 - RNA, 22 nt, Artificial Sequence
  Homo sapiens
cugccaauuccauaggucacag

SEQ ID NO: 27 - protein, 13 aa, Artificial Sequence
  Unknown
ELYENKPRRPYIL

SEQ ID NO: 28 - RNA, 10 nt, Artificial Sequence
  Homo sapiens
nnacuggcnn

SEQ ID NO: 29 - RNA, 10 nt, Artificial Sequence
  Homo sapiens
nnagcccunn

SEQ ID NO: 30 - DNA, 23 bp, Artificial Sequence
  Homo sapiens
tcacgaaggtgtacatgctttgg

SEQ ID NO: 31 - DNA, 23 bp, Artificial Sequence
  Homo sapiens
gatcctcaacgtgcaaaagaagc

SEQ ID NO: 32 - DNA, 20 bp, Artificial Sequence
  Homo sapiens
taaaggaacagacgggctct

SEQ ID NO: 33 - DNA, 20 bp, Artificial Sequence
  Homo sapiens
ccatagacgccatcattcac

SEQ ID NO: 34 - DNA, 20 bp, Artificial Sequence
  Homo sapiens
ggaaccgtagactcgctcat

SEQ ID NO: 35 - DNA, 20 bp, Artificial Sequence
  Homo sapiens
tcggagtgagcgtttatttg

SEQ ID NO: 36 - DNA, 20 bp, Artificial Sequence
  Homo sapiens
catcaatggaaatcccatca

SEQ ID NO: 37 - DNA, 20 bp, Artificial Sequence
  Homo sapiens
ttctccatggtggtgaagac

SEQ ID NO: 38 - DNA, 21 bp, Artificial Sequence
  Homo sapiens
gcaguggacauagaagagatt

SEQ ID NO: 39 - DNA, 21 bp, Artificial Sequence
  Homo sapiens
cgcuuuaacuggaagcucutt

SEQ ID NO: 40 - DNA, 21 bp, Artificial Sequence
  Homo sapiens
cgauguucguggacauucutt

SEQ ID NO: 41 - DNA, 21 bp, Artificial Sequence
  Homo sapiens
caacaguacuuaccgaugutt

SEQ ID NO: 42 - DNA, 21 bp, Artificial Sequence
  Homo sapiens
caagucaccuguuaccucatt

SEQ ID NO: 43 - DNA, 21 bp, Artificial Sequence
  Homo sapiens
gcuaagucuuuuccacagutt

SEQ ID NO: 44 - DNA, 21 bp, Artificial Sequence
  Homo sapiens
caacagaucagagaacaaatt

SEQ ID NO: 45 - DNA, 21 bp, Artificial Sequence
  Homo sapiens
ggauauuggaggcuagucatt

SEQ ID NO: 46 - DNA, 21 bp, Artificial Sequence
  Homo sapiens
cucuuaugguaaccaaugatt

SEQ ID NO: 47 - DNA, 21 bp, Artificial Sequence
  Homo sapiens
guaccugcgggauuacucatt

SEQ ID NO: 48 - DNA, 21 bp, Artificial Sequence
  Homo sapiens
gcaaugcuugcaaaacucatt

SEQ ID NO: 49 - DNA, 21 bp, Artificial Sequence
  Homo sapiens
cgaaacuugucaacgggaatt

SEQ ID NO: 50 - DNA, 21 bp, Artificial Sequence
  Homo sapiens
ggaggaaucucgaaugcgatt

SEQ ID NO: 51 - DNA, 21 bp, Artificial Sequence
  Homo sapiens
ccagcgauacaacuccuautt

SEQ ID NO: 52 - DNA, 21 bp, Artificial Sequence
  Homo sapiens
ggaagaacgucuugcacaatt

SEQ ID NO: 53 - DNA, 21 bp, Artificial Sequence
  Homo sapiens
gaacaaaauagaccucuuatt

SEQ ID NO: 54 - DNA, 21 bp, Artificial Sequence
  Homo sapiens
gcaucuguccgaucgcucutt

SEQ ID NO: 55 - DNA, 21 bp, Artificial Sequence
  Homo sapiens
caacgggacuauuaugauatt

SEQ ID NO: 56 - DNA, 21 bp, Artificial Sequence
  Homo sapiens
caacgggacuauuaugauatt

SEQ ID NO: 57 - DNA, 21 bp, Artificial Sequence
  Homo sapiens
gaacgaguacagcaauauatt

SEQ ID NO: 58 - DNA, 21 bp, Artificial Sequence
  Homo sapiens
acaggucuuuaucaaagcatt

SEQ ID NO: 59 - DNA, 21 bp, Artificial Sequence
  Homo sapiens
agaucuuugucaagguguatt

SEQ ID NO: 60 - DNA, 21 bp, Artificial Sequence
  Homo sapiens
ggcgaaugcuccugacuaatt

SEQ ID NO: 61 - DNA, 21 bp, Artificial Sequence
  Homo sapiens
ggacggaagcgaaaaucgatt

SEQ ID NO: 62 - DNA, 21 bp, Artificial Sequence
  Homo sapiens
aggucaaugugacuuuccatt

SEQ ID NO: 63 - DNA, 21 bp, Artificial Sequence
  Homo sapiens
ccuggacaguuugaagcaatt

SEQ ID NO: 64 - DNA, 21 bp, Artificial Sequence
  Homo sapiens
gaucguuugagagaacaaatt

SEQ ID NO: 65 - DNA, 21 bp, Artificial Sequence
  Homo sapiens
gccuaggaguuauccucuatt

SEQ ID NO: 66 - DNA, 21 bp, Artificial Sequence
  Homo sapiens
gcaacagaaucgagcaagatt

SEQ ID NO: 67 - DNA, 21 bp, Artificial Sequence
  Homo sapiens
ggaugacgagcugaacuuutt

SEQ ID NO: 68 - DNA, 21 bp, Artificial Sequence
  Homo sapiens
cagguaguguaagcccucatt

SEQ ID NO: 69 - DNA, 21 bp, Artificial Sequence
  Homo sapiens
cccuccuacuauuacccgutt

SEQ ID NO: 70 - DNA, 21 bp, Artificial Sequence
  Homo sapiens
gauuauccuuguacuuugatt

SEQ ID NO: 71 - DNA, 21 bp, Artificial Sequence
  Homo sapiens
gauuacuucuugacuaucatt

SEQ ID NO: 72 - DNA, 21 bp, Artificial Sequence
  Homo sapiens
gcuccucuguuaccacagatt

SEQ ID NO: 73 - DNA, 21 bp, Artificial Sequence
  Homo sapiens
guccgagugugguucuguatt

SEQ ID NO: 74 - DNA, 21 bp, Artificial Sequence
  Homo sapiens
cuucgacaguacuuccggatt

SEQ ID NO: 75 - DNA, 21 bp, Artificial Sequence
  Homo sapiens
gcucaucgacaucaaaguutt

SEQ ID NO: 76 - DNA, 21 bp, Artificial Sequence
  Homo sapiens
cuuuuaauguuaaacuacatt

SEQ ID NO: 77 - DNA, 21 bp, Artificial Sequence
  Homo sapiens
caauggaacaaucaaaguutt

SEQ ID NO: 78 - DNA, 21 bp, Artificial Sequence
  Homo sapiens
cggaaucaaugauccuguatt

SEQ ID NO: 79 - DNA, 21 bp, Artificial Sequence
  Homo sapiens
aagucaacauagcccgaaatt

SEQ ID NO: 80 - DNA, 21 bp, Artificial Sequence
  Homo sapiens
caaugauucuuacccucaatt

SEQ ID NO: 81 - DNA, 21 bp, Artificial Sequence
  Homo sapiens
guuucaucuggguucgcuatt

SEQ ID NO: 82 - DNA, 21 bp, Artificial Sequence
  Homo sapiens
guccuaucaccguaucuuatt

SEQ ID NO: 83 - DNA, 21 bp, Artificial Sequence
  Homo sapiens
gcggaaaagguaucccucatt

SEQ ID NO: 84 - DNA, 21 bp, Artificial Sequence
  Homo sapiens
ggcuguacauaguccuuuutt

SEQ ID NO: 85 - DNA, 21 bp, Artificial Sequence
  Homo sapiens
cugccgcaacaauaagaaatt

SEQ ID NO: 86 - DNA, 21 bp, Artificial Sequence
  Homo sapiens
cauugguuuugaugaguautt

SEQ ID NO: 87 - DNA, 21 bp, Artificial Sequence
  Homo sapiens
gguguaauaauguccuuuatt

SEQ ID NO: 88 - DNA, 21 bp, Artificial Sequence
  Homo sapiens
gagcagagaucauaaccaatt

SEQ ID NO: 89 - DNA, 21 bp, Artificial Sequence
  Homo sapiens
ggaucacgcagcucaagautt

SEQ ID NO: 90 - DNA, 21 bp, Artificial Sequence
  Homo sapiens
ggugcucucgguugcacaatt

SEQ ID NO: 91 - DNA, 21 bp, Artificial Sequence
  Homo sapiens
cagcaaaccuuugaccagatt

SEQ ID NO: 92 - DNA, 21 bp, Artificial Sequence
  Homo sapiens
ccaguucgaccugaacaaatt

SEQ ID NO: 93 - DNA, 21 bp, Artificial Sequence
  Homo sapiens
cagccucaccaagcacaaatt

SEQ ID NO: 94 - DNA, 21 bp, Artificial Sequence
  Homo sapiens
agaggcuacuuugaaaacatt

SEQ ID NO: 95 - DNA, 21 bp, Artificial Sequence
  Homo sapiens
caagucaccuguuaccucatt

SEQ ID NO: 96 - DNA, 21 bp, Artificial Sequence
  Homo sapiens
acacucuuguggucaagaatt

SEQ ID NO: 97 - DNA, 21 bp, Artificial Sequence
  Homo sapiens
gaugaagaaugauaagucatt

SEQ ID NO: 98 - DNA, 21 bp, Artificial Sequence
  Homo sapiens
caacgggacuauuaugauatt

SEQ ID NO: 99 - DNA, 21 bp, Artificial Sequence
  Homo sapiens
cccggauacuucugaagaatt

SEQ ID NO: 100 - DNA, 21 bp, Artificial Sequence
  Homo sapiens
gaacgaguacagcaauauatt

SEQ ID NO: 101 - DNA, 21 bp, Artificial Sequence
  Homo sapiens
guucauccauaaguacauutt

SEQ ID NO: 102 - DNA, 21 bp, Artificial Sequence
  Homo sapiens
agaucuuugucaagguguatt

SEQ ID NO: 103 - DNA, 21 bp, Artificial Sequence
  Homo sapiens
gacauuggaucauacuaaatt

SEQ ID NO: 104 - DNA, 21 bp, Artificial Sequence
  Homo sapiens
ggcgaaugcuccugacuaatt

SEQ ID NO: 105 - DNA, 21 bp, Artificial Sequence
  Homo sapiens
gcgcaagcuuaagaacuuutt

SEQ ID NO: 106 - DNA, 21 bp, Artificial Sequence
  Homo sapiens
gcucaucgacaucaaaguutt

SEQ ID NO: 107 - DNA, 21 bp, Artificial Sequence
  Homo sapiens
ccauauauccgaaugaccatt

SEQ ID NO: 108 - DNA, 21 bp, Artificial Sequence
  Homo sapiens
cggaaucaaugauccuguatt

SEQ ID NO: 109 - DNA, 21 bp, Artificial Sequence
  Homo sapiens
gcaucgaggagacuaacaatt

SEQ ID NO: 110 - DNA, 21 bp, Artificial Sequence
  Homo sapiens
caaugauucuuacccucaatt

SEQ ID NO: 111 - DNA, 21 bp, Artificial Sequence
  Homo sapiens
caaccuuauuaucauugaatt

SEQ ID NO: 112 - DNA, 21 bp, Artificial Sequence
  Homo sapiens
guuucaucuggguucgcuatt

SEQ ID NO: 113 - DNA, 21 bp, Artificial Sequence
  Homo sapiens
gcaucagcucacaacaaaatt

SEQ ID NO: 114 - DNA, 21 bp, Artificial Sequence
  Homo sapiens
guccuaucaccguaucuuatt

SEQ ID NO: 115 - DNA, 21 bp, Artificial Sequence
  Homo sapiens
agauacugguauugcucgatt

SEQ ID NO: 116 - DNA, 21 bp, Artificial Sequence
  Homo sapiens
uggucucaaugacaguagatt

SEQ ID NO: 117 - DNA, 21 bp, Artificial Sequence
  Homo sapiens
ggcuguacauaguccuuuutt

SEQ ID NO: 118 - DNA, 21 bp, Artificial Sequence
  Homo sapiens
uguggacugagguacccaatt

SEQ ID NO: 119 - DNA, 21 bp, Artificial Sequence
  Homo sapiens
cugccgcaacaauaagaaatt

SEQ ID NO: 120 - DNA, 21 bp, Artificial Sequence
  Homo sapiens
ggaucaugcuaaaaggagatt

SEQ ID NO: 121 - DNA, 21 bp, Artificial Sequence
  Homo sapiens
cauugguuuugaugaguautt

SEQ ID NO: 122 - DNA, 21 bp, Artificial Sequence
  Homo sapiens
agggcuaucugguaucugutt

SEQ ID NO: 123 - DNA, 21 bp, Artificial Sequence
  Homo sapiens
gguguaauaauguccuuuatt

SEQ ID NO: 124 - DNA, 21 bp, Artificial Sequence
  Homo sapiens
gaauaaccguugguuuaautt

SEQ ID NO: 125 - DNA, 21 bp, Artificial Sequence
  Homo sapiens
ggaacacuaugaugaguuutt

SEQ ID NO: 126 - DNA, 21 bp, Artificial Sequence
  Homo sapiens
ggugcucucgguugcacaatt

SEQ ID NO: 127 - DNA, 21 bp, Artificial Sequence
  Homo sapiens
ccaacuaccugaacgaugatt

SEQ ID NO: 128 - DNA, 21 bp, Artificial Sequence
  Homo sapiens
cagcaaaccuuugaccagatt

SEQ ID NO: 129 - DNA, 21 bp, Artificial Sequence
  Homo sapiens
gcuauuuccagcgaauauatt

SEQ ID NO: 130 - DNA, 21 bp, Artificial Sequence
  Homo sapiens
ggaggaaucucgaaugcgatt

SEQ ID NO: 131 - DNA, 21 bp, Artificial Sequence
  Homo sapiens
ggagaaagaaucaacggaatt

SEQ ID NO: 132 - DNA, 21 bp, Artificial Sequence
  Homo sapiens
ggacggaagcgaaaaucgatt

SEQ ID NO: 133 - DNA, 21 bp, Artificial Sequence
  Homo sapiens
gaacuucaauggaaaacautt

SEQ ID NO: 134 - DNA, 21 bp, Artificial Sequence
  Homo sapiens
gaucguuugagagaacaaatt

SEQ ID NO: 135 - DNA, 21 bp, Artificial Sequence
  Homo sapiens
cagguaguguaagcccucatt

SEQ ID NO: 136 - DNA, 21 bp, Artificial Sequence
  Homo sapiens
agguuacagaggacucagatt

SEQ ID NO: 137 - DNA, 21 bp, Artificial Sequence
  Homo sapiens
gaaccgucgcuacaagacatt

SEQ ID NO: 138 - DNA, 21 bp, Artificial Sequence
  Homo sapiens
cccuccuacuauuacccgutt

SEQ ID NO: 139 - DNA, 21 bp, Artificial Sequence
  Homo sapiens
ggagauaugcaaagcagaatt

SEQ ID NO: 140 - DNA, 21 bp, Artificial Sequence
  Homo sapiens
guccgagugugguucuguatt

SEQ ID NO: 141 - DNA, 21 bp, Artificial Sequence
  Homo sapiens
agaggacccagauugucuatt

SEQ ID NO: 142 - DNA, 21 bp, Artificial Sequence
  Homo sapiens
aagucaacauagcccgaaatt

SEQ ID NO: 143 - DNA, 21 bp, Artificial Sequence
  Homo sapiens
gcaaagauagcgagaaauatt

SEQ ID NO: 144 - DNA, 21 bp, Artificial Sequence
  Homo sapiens
ggaucacgcagcucaagautt

SEQ ID NO: 145 - DNA, 21 bp, Artificial Sequence
  Homo sapiens
ccaguucgaccugaacaaatt

SEQ ID NO: 146 - DNA, 21 bp, Artificial Sequence
  Homo sapiens
gaccuauacucauucugcatt

SEQ ID NO: 147 - DNA, 21 bp, Artificial Sequence
  Homo sapiens
guuucgaccucgauccagatt

SEQ ID NO: 148 - DNA, 21 bp, Artificial Sequence
  Homo sapiens
cagccucaccaagcacaaatt

SEQ ID NO: 149 - DNA, 21 bp, Artificial Sequence
  Homo sapiens
cgaccaucauggaaaacautt

SEQ ID NO: 150 - DNA, 21 bp, Artificial Sequence
  Homo sapiens
cgcuuuaacuggaagcucutt

SEQ ID NO: 151 - DNA, 21 bp, Artificial Sequence
  Homo sapiens
gaaacugcaugagaagacatt

SEQ ID NO: 152 - DNA, 21 bp, Artificial Sequence
  Homo sapiens
cgauguucguggacauucutt

SEQ ID NO: 153 - DNA, 21 bp, Artificial Sequence
  Homo sapiens
cacuuuccuggguauuguatt

SEQ ID NO: 154 - DNA, 21 bp, Artificial Sequence
  Homo sapiens
caacaguacuuaccgaugutt

SEQ ID NO: 155 - DNA, 21 bp, Artificial Sequence
  Homo sapiens
gccuugcuccgaaccuucatt

SEQ ID NO: 156 - DNA, 21 bp, Artificial Sequence
  Homo sapiens
gauuauccuuguacuuugatt

SEQ ID NO: 157 - DNA, 21 bp, Artificial Sequence
  Homo sapiens
gaacagcugggaaccuucatt

SEQ ID NO: 158 - DNA, 21 bp, Artificial Sequence
  Homo sapiens
cuucgacaguacuuccggatt

SEQ ID NO: 159 - DNA, 21 bp, Artificial Sequence
  Homo sapiens
ggaguucuuugaugagaaatt

SEQ ID NO: 160 - DNA, 21 bp, Artificial Sequence
  Homo sapiens
ggaccaagcccuaaaggaatt

SEQ ID NO: 161 - DNA, 21 bp, Artificial Sequence
  Homo sapiens
gcaguggacauagaagagatt

SEQ ID NO: 162 - DNA, 21 bp, Artificial Sequence
  Homo sapiens
cgauaugcuuuucuacguutt

SEQ ID NO: 163 - DNA, 21 bp, Artificial Sequence
  Homo sapiens
ccagcgauacaacuccuautt

SEQ ID NO: 164 - DNA, 21 bp, Artificial Sequence
  Homo sapiens
gcaggcacaugguuccucutt

SEQ ID NO: 165 - DNA, 21 bp, Artificial Sequence
  Homo sapiens
aggucaaugugacuuuccatt

SEQ ID NO: 166 - DNA, 21 bp, Artificial Sequence
  Homo sapiens
ccaaguccaaugcaccugutt

SEQ ID NO: 167 - DNA, 21 bp, Artificial Sequence
  Homo sapiens
ccuggacaguuugaagcaatt

SEQ ID NO: 168 - DNA, 21 bp, Artificial Sequence
  Homo sapiens
gcuuccucuuggaccucaatt

SEQ ID NO: 169 - DNA, 21 bp, Artificial Sequence
  Homo sapiens
gcggaaaagguaucccucatt

SEQ ID NO: 170 - DNA, 21 bp, Artificial Sequence
  Homo sapiens
ccaacaaggaagacgaaaatt

SEQ ID NO: 171 - DNA, 21 bp, Artificial Sequence
  Homo sapiens
cccuguucauuauaagucutt

SEQ ID NO: 172 - DNA, 21 bp, Artificial Sequence
  Homo sapiens
ggauauuggaggcuagucatt

SEQ ID NO: 173 - DNA, 21 bp, Artificial Sequence
  Homo sapiens
gacuauugauaccaaaacatt

SEQ ID NO: 174 - DNA, 21 bp, Artificial Sequence
  Homo sapiens
gcaaugcuugcaaaacucatt

SEQ ID NO: 175 - DNA, 21 bp, Artificial Sequence
  Homo sapiens
cauugguguugacaauccatt

SEQ ID NO: 176 - DNA, 21 bp, Artificial Sequence
  Homo sapiens
guugcaaacuuaucgaggatt

SEQ ID NO: 177 - DNA, 21 bp, Artificial Sequence
  Homo sapiens
cucuuaugguaaccaaugatt

SEQ ID NO: 178 - DNA, 21 bp, Artificial Sequence
  Homo sapiens
aaaugcaacccaaaaaguatt

SEQ ID NO: 179 - DNA, 21 bp, Artificial Sequence
  Homo sapiens
guaccugcgggauuacucatt

SEQ ID NO: 180 - DNA, 21 bp, Artificial Sequence
  Homo sapiens
ccacccuacuauguacugutt

SEQ ID NO: 181 - DNA, 21 bp, Artificial Sequence
  Homo sapiens
gcuaagucuuuuccacagutt

SEQ ID NO: 182 - DNA, 21 bp, Artificial Sequence
  Homo sapiens
caguuacuuucaaaacucutt

SEQ ID NO: 183 - DNA, 21 bp, Artificial Sequence
  Homo sapiens
caacagaucagagaacaaatt

SEQ ID NO: 184 - DNA, 21 bp, Artificial Sequence
  Homo sapiens
gccugaaucuugucccaaatt

SEQ ID NO: 185 - DNA, 21 bp, Artificial Sequence
  Homo sapiens
gaagaagacucuguacucatt

SEQ ID NO: 186 - DNA, 21 bp, Artificial Sequence
  Homo sapiens
cgaaacuugucaacgggaatt

SEQ ID NO: 187 - DNA, 21 bp, Artificial Sequence
  Homo sapiens
agaaagcuuugggcaaauatt

SEQ ID NO: 188 - DNA, 21 bp, Artificial Sequence
  Homo sapiens
ggaagaacgucuugcacaatt

SEQ ID NO: 189 - DNA, 21 bp, Artificial Sequence
  Homo sapiens
gaacaaaauagaccucuuatt

SEQ ID NO: 190 - DNA, 21 bp, Artificial Sequence
  Homo sapiens
cuguggaaaagauaccguatt

SEQ ID NO: 191 - DNA, 21 bp, Artificial Sequence
  Homo sapiens
gcccacuauucucagcucatt

SEQ ID NO: 192 - DNA, 21 bp, Artificial Sequence
  Homo sapiens
gcaucuguccgaucgcucutt

SEQ ID NO: 193 - DNA, 21 bp, Artificial Sequence
  Homo sapiens
gggacacugucaaccucuatt

SEQ ID NO: 194 - DNA, 21 bp, Artificial Sequence
  Homo sapiens
caucaagcccuguaagucutt

SEQ ID NO: 195 - DNA, 21 bp, Artificial Sequence
  Homo sapiens
ugaagauaaucgagaaauatt

SEQ ID NO: 196 - DNA, 21 bp, Artificial Sequence
  Homo sapiens
acaggucuuuaucaaagcatt

SEQ ID NO: 197 - DNA, 21 bp, Artificial Sequence
  Homo sapiens
gacucagaguaacaacgcatt

SEQ ID NO: 198 - DNA, 21 bp, Artificial Sequence
  Homo sapiens
gccuaggaguuauccucuatt

SEQ ID NO: 199 - DNA, 21 bp, Artificial Sequence
  Homo sapiens
cuaacuacccagacccuuatt

SEQ ID NO: 200 - DNA, 21 bp, Artificial Sequence
  Homo sapiens
gcaacagaaucgagcaagatt

SEQ ID NO: 201 - DNA, 21 bp, Artificial Sequence
  Homo sapiens
cccugaagguuaccucuuutt

SEQ ID NO: 202 - DNA, 21 bp, Artificial Sequence
  Homo sapiens
ggaugacgagcugaacuuutt

SEQ ID NO: 203 - DNA, 21 bp, Artificial Sequence
  Homo sapiens
gauuacuucuugacuaucatt

SEQ ID NO: 204 - DNA, 21 bp, Artificial Sequence
  Homo sapiens
cucccgcaguugaacaucatt

SEQ ID NO: 205 - DNA, 21 bp, Artificial Sequence
  Homo sapiens
cucugauugucaccucuuatt

SEQ ID NO: 206 - DNA, 21 bp, Artificial Sequence
  Homo sapiens
gcuccucuguuaccacagatt

SEQ ID NO: 207 - DNA, 21 bp, Artificial Sequence
  Homo sapiens
cgcucaccaacagcagaaatt

SEQ ID NO: 208 - DNA, 21 bp, Artificial Sequence
  Homo sapiens
cuuuuaauguuaaacuacatt

SEQ ID NO: 209 - DNA, 21 bp, Artificial Sequence
  Homo sapiens
gaacuucacaaguuccucatt

SEQ ID NO: 210 - DNA, 21 bp, Artificial Sequence
  Homo sapiens
caauggaacaaucaaaguutt

SEQ ID NO: 211 - DNA, 21 bp, Artificial Sequence
  Homo sapiens
ggauuacagaaacuccuautt

SEQ ID NO: 212 - DNA, 21 bp, Artificial Sequence
  Homo sapiens
gagcagagaucauaaccaatt

SEQ ID NO: 213 - DNA, 21 bp, Artificial Sequence
  Homo sapiens
ggcugaauguaacagaggatt

SEQ ID NO: 214 - DNA, 21 bp, Artificial Sequence
  Homo sapiens
ccguagacucgcucaucuctt

SEQ ID NO: 215 - DNA, 21 bp, Artificial Sequence
  Homo sapiens
gcuaacuuauucuacuccgtt

SEQ ID NO: 216 - DNA, 21 bp, Artificial Sequence
  Homo sapiens
gggaauagucagagggauctt

SEQ ID NO: 217 - DNA, 21 bp, Artificial Sequence
  Homo sapiens
acaucguccacuuccaguatt

SEQ ID NO: 218 - DNA, 21 bp, Artificial Sequence
  Homo sapiens
ggaccucccugugaaugautt

SEQ ID NO: 219 - DNA, 21 bp, Artificial Sequence
  Homo sapiens
cagauggauuauuagcugatt

SEQ ID NO: 220 - DNA, 21 bp, Artificial Sequence
  Homo sapiens
ccgggaaaguuugacugcatt

SEQ ID NO: 221 - DNA, 21 bp, Artificial Sequence
  Homo sapiens
ccacgaccagcuuaaagaatt

SEQ ID NO: 222 - DNA, 21 bp, Artificial Sequence
  Homo sapiens
gacuuucacuuccgccuuatt

SEQ ID NO: 223 - DNA, 21 bp, Artificial Sequence
  Homo sapiens
gaugacuuuugauagugaatt

SEQ ID NO: 224 - DNA, 21 bp, Artificial Sequence
  Homo sapiens
gcauguaucugcuugauautt

SEQ ID NO: 225 - DNA, 21 bp, Artificial Sequence
  Homo sapiens
gcuugcaguuaauaucauutt

SEQ ID NO: 226 - DNA, 21 bp, Artificial Sequence
  Homo sapiens
cacucgcaguuaauaucautt

SEQ ID NO: 227 - DNA, 21 bp, Artificial Sequence
  Homo sapiens
cgaugaacauguuagacautt

SEQ ID NO: 228 - DNA, 21 bp, Artificial Sequence
  Homo sapiens
gcccucuguuggauaucuatt